(19) United States
(12) Patent Application Publication
(10) Pub. No.: US 2010/0296968 A1
(43) Pub. Date: Nov. 25, 2010

(54) SANITIZING DEVICES FOR NEEDLELESS MEDICAL FITTINGS

(75) Inventor: Timothy B. CADY, Encinitas, CA (US)

Correspondence Address: BioTechnology Law Group;12707 High Bluff Drive, Suite 200, San Diego, CA, 92130-2037, US

(73) Assignee: ZINC MEDICAL, INC., Solana Beach, CA (US)

(21) Appl. No.: 12/783,372

(22) Filed: May 19, 2010

Related U.S. Application Data
(60) Provisional application No. 61/179,699, filed on May 19, 2009.

(51) Int. Cl. A61L 2/16; B65D 85/00

(52) U.S. Cl. 422/28; 206/349

(57) ABSTRACT

Single-use devices configured to sanitize accessible surfaces of needleless medical valves at risk of contamination with infectious agents are described, as are methods for making and using such devices. Such devices include a sanitizing element securely attached to an inner surface of at least one of the two or more layers that make up the container inside of which the sanitizing element is stored until it is ready to be used. Examples of containers include laminated foil pouches or packets.

### RELATED APPLICATION

This patent application claims priority to and the benefit of U.S. provisional patent application Ser. No. 61/179,699, filed on 19 May 2009, the contents of which is hereby incorporated by reference in its entirety for any and all purposes.

### TECHNICAL FIELD

This invention concerns small disposable, single-purpose devices useful for sanitizing needleless valves on medical fittings, particularly those surfaces of such valves that are or may be at risk of contamination with infectious agents.

### BACKGROUND OF THE INVENTION

1. Introduction

The following description includes information that may be useful in understanding the present invention. It is not an admission that any such information is prior art, or relevant, to the presently claimed inventions, or that any publication specifically or implicitly referenced is prior art.

2. Background

Exposure to infectious agents (e.g., pathogenic bacteria, viruses, fungi, etc.) in medical settings is a matter of serious concern. One route of exposure to such agents is the opening made in skin provided by the bore of needle, canula, or other similar device used to provide access to a patient's vasculature. It is known that patients whose skin has been compromised in this way are at increased risk for developing serious blood stream infections. In the United States alone, approximately 300,000 blood stream infections per year result from the installation and use of peripheral intravenous catheters (PIVC), and more than 80,000 blood stream infections are associated with the use central venous catheters (CVC). All told, in the U.S. approximately 20,000 patients die annually from hospital acquired infections that result from PIVC and CVC use. Costs associated with the care and treatment of patients that develop infections due to PIVC and CVC use exceed $2.7 billion.

In hospital settings today, occupational health and safety regulations designed reduce the risk to health care workers from needle prick and similar injuries have resulted in the deployment of needleless medical valves whenever possible. Currently, more than 500 million needleless valves are used annually in hospitals throughout the U.S. Needleless valves are used primarily in conjunction with PIVC and CVC devices, which may contain from as few as one to as many as 3, 4, 5, or more needleless valves. FIG. 1 illustrates an example of a representative medical valve in use today.

The widespread use of needleless valves in acute medicine has contributed to a marked increase in the incidence of hospital acquired infections (HAIs), particularly blood stream infections. To reduce the risk of infection from a contaminated needleless valve, standard practice today requires that a nurse or other health care worker clean the surface of a needleless valve by rubbing it with a sterile alcohol swab or wipe immediately prior to making a connection to the valve, for example, attaching a syringe to the valve to deliver a medication via a PIVC already connected to a patient. Given the magnitude of the mortality and morbidity associated with HAIs and the large number of blood stream infections that result from PIVC and CVC use, a long-recognized yet significant unmet need exists for articles or devices that can be used to reduce or eliminate the risk of initiating an HAI merely by accessing a patient's vasculature through a needleless valve component of a PIVC or CVC inserted into a blood vessel of a patient. The present invention addresses this need through the provision of disposable, single-use devices that can be quickly and easily used to sanitize needleless medical valves.

3. Definitions

Before describing the instant invention in detail, several terms used in the context of the present invention will be defined. In addition to these terms, others are defined elsewhere in the specification, as necessary. Unless otherwise expressly defined herein, terms of art used in this specification will have their art-recognized meanings.

An “aqueous solution” refers to a water-based solution capable of dissolving or dispersing one or more other substances, or solutes (i.e., the substance(s) dissolved in the solvent). A “solution” is a homogeneous mixture of at least one substance in a liquid. In the context of this invention, “aqueous solvents” can also include other liquids, including organic liquids, such as alcohols and/or oils.

An “infectious agent” refers to any organism capable of infecting another organism. Such agents include many bacteria, viruses, and fungi.

A “patentable” composition, process, machine, or article of manufacture according to the invention means that the subject matter at issue satisfies all statutory requirements for patentability at the time the analysis is performed. For example, with regard to novelty, non-obviousness, or the like, if later investigation reveals that one or more claims encompass one or more embodiments that would negate novelty, non-obviousness, etc., the claim(s), being limited by definition to “patentable” embodiments, specifically excludes the unpatentable embodiment(s). Also, the claims appended hereto are to be interpreted both to provide the broadest reasonable scope, as well as to preserve their validity. Furthermore, if one or more of the statutory requirements for patentability are amended or if the standards change for assessing whether a particular statutory requirement for patentability is satisfied from the time this application is filed or issues as a patent to a time the validity of one or more of the appended claims is questioned, the claims are to be interpreted in a way that (1) preserves their validity and (2) provides the broadest reasonable interpretation under the circumstances.

A “plurality” means more than one.

In a “suspension” solid particles are dispersed in a liquid. The term “colloidal” refers to a state of subdivision, which, in the context of solutions, means that molecules or particles dispersed in the liquid have at least in one direction a dimension roughly between 1 nm and 1 μm. It is not necessary for all three dimensions to be in the colloidal range. A “colloidal dispersion” is a system in which particles of colloidal size of any nature (e.g. solid, liquid or gas) are dispersed in a continuous phase of a different composition (or state). In an “emulsion” liquid droplets and/or liquid crystals are dispersed in another liquid. An emulsion may be denoted by the symbol “O/W” if the continuous phase (i.e., is an aqueous solution) and by “W/O” if the continuous phase is an organic liquid.

### SUMMARY OF THE INVENTION

It is an object of this invention to provide patentable single-use sanitizing devices or articles that can be used to effectively and efficiently sanitize, and preferably sterilize, exposed surfaces of needleless medical valves, particularly the accessible surface of the valve stems of needleless valves of medical fittings, particularly those surfaces that may become contaminated with infectious agents. In the context of the invention, “sanitize” encompasses cleaning, disinfecting, and/or sterilizing.

Thus, one aspect of the invention concerns patentable single-use sanitizing articles configured to sanitize needleless valves of medical fittings. Sanitizing devices, or articles, according to the invention are sealed, sterilized single-use devices that, once unsealed and used to sanitize a needleless medical vale, can be disposed of. The sanitizing devices of the invention typically comprise a sanitizing element associated with a sealed, single use multi-layered container that can be easily opened to expose the sanitizing element. Unlike conventional IPA wipes, the sanitizing element of an article according to the invention is not removed from its container prior to use. Instead, the sanitizing element remains attached or otherwise associated with at least a portion of the container by being fixedly associated with at least part of an inner surface of a portion or region of one or more layers of the container. When a container is unsealed (i.e., opened), for example, by being torn open or peeled apart just prior to use by a health care worker, the sanitizing element remains attached to a portion of the multi-layered container so that the sanitizing element is presented or made available in a manner that allows the health care worker to easily bring the sanitizing element into sanitizing association with the needleless medical valve to be sanitized, particularly those exposed surfaces of the valve that are also likely to contact fluid that will pass through the valve and into the patient from a fluid delivery device or fluid reservoir positioned upstream of the valve (e.g., a syringe, an I.V. bag, etc.). In other words, the sanitizing element is configured to accommodate the three dimensional structure of a needleless medical valve so as to allow those surfaces of the valve that are likely to be contaminated and which may form part of the fluid communication pathway between an external fluid source and the patient's blood stream to be easily sanitized immediately prior to attachment of a fluid reservoir to the needleless medical valve.

In the devices of the invention, a sanitizing element comprises a substrate and a sanitizing reagent dispersed in the substrate prior to opening the device prior to use. In many preferred embodiments, the sanitizing reagent is dispersed in the substrate at the time the device is manufactured and before the pouch is sealed. Of course, the invention also includes embodiments where the sanitizing reagent is released for dispersion into the substrate post-manufacture or post-sealing, but prior to the time the device is brought into contact with the needleless valve to be sanitized.

The substrate of a sanitizing element includes a sanitizing region configured to engage an accessible surface of a valve stem of a needleless medical valve so as to expose the accessible surface, and any infectious agents residing thereon, to the sanitizing reagent. Also, because valve surfaces may be contaminated with microorganisms that form a biofilm (i.e., a matrix of microorganisms and extracellular material attached to a surface, which enables the microorganisms, typically bacteria and/or fungi, to adhere to a surface and carry out certain biochemical processes), the sanitizing element also preferably has sufficient mechanical integrity to allow its use to disrupt the biofilm, such as can occur by rotating, twisting, or otherwise moving the sanitizing element in relation to the needleless medical when the sanitizing device is brought into contact with the valve.

In some embodiments, the substrate of the sanitizing element comprises a single layer of substantially uniform thickness, whereas in others, it comprises a single layer of varying thickness that may have contours or features designed to enhance sanitizing contact with the surface(s) to be cleaned. Alternatively, a sanitizing element substrate can be made from a plurality of layers, each of which may be of substantially uniform or varying thickness(es), and which together are integrated to form a substrate of desired thickness(es) and surface contour(s). In devices that have a multi-layered sanitizing element, the substrate used to form each layer may be of the same or different material, may be of the same or different dimensions (length, width and/or thickness), and may or may not contain a sanitizing reagent. When two or more layers each contain a sanitizing reagent, the reagent may the same or different, although preferably they are compatible such that one reagent does not appreciably degrade the sanitizing capacity of the other. Additionally, in some embodiments of multi-layer devices, one or more of the layers may be physically separated from the other layer(s) by an impermeable, semi-permeable, or permeable barrier.

In preferred embodiments, the substrate used to form the sanitizing element is any suitable absorbent material that is pliable, fibrous, and/or porous, or combination of materials that can be wetted and/or impregnated with a sanitizing reagent. Such materials include those that are synthetic or naturally occurring, and they may be of homogeneous or heterogeneous composition, and include materials where natural and synthetic materials are blended. Preferred synthetic materials include fibers, foams, and gel compositions. Preferred natural materials include those derived from fibrous materials such as cotton and silk, which materials can be spun and woven, as well as materials such as natural sponges. With respect to synthetic fibrous materials, those having directly oriented fibers are particularly preferred. In embodiments wherein the sanitizing element is comprised of two or more layers, each layer can be formed from a material that is the same as or different from the material used to form the other layer(s), and each layer may contain the same, different, or even no, sanitizing reagent (although at least one layer will have a sanitizing reagent dispersed therein prior to engaging the surface of the needleless valve to be sanitized). Also, even when substrates for different layers are formed from the same material, they may be configured differently. For example, in one particularly preferred embodiment that employs a sanitizing element having two layers, the substrate for the upper and lower layers is formed from a natural material such as woven or spun cotton. The upper layer is smaller than and centered on the lower layer, which is attached (for example, by an adhesive) to the inner surface of the container. In another embodiment that employs a sanitizing element having two layers, the substrate for the upper layer is formed from an absorbent synthetic material (e.g., an absorbent synthetic material having directionally oriented fibers or a synthetic foam pad) that also has mild abrasive characteristics, while the substrate for the lower layer is an absorbent pad formed from a natural material such as woven or spun cotton. When sanitizing element substrates are made from two or more layers, preferably the layers are adhered or otherwise associated at an interface using a suitable adhesive or other joining material to allow the layers to remain associated during use.

In embodiments where the sanitizing element includes a material having abrasive characteristics in order to achieve improved sanitizing of the potentially contaminated exposed surface(s) of a needleless medical fitting or valve, such abrasive layer or material may or may not comprise a sanitizing reagent dispersed therein during manufacture; however, any such layer allows sanitizing reagents disposed in other layers of the sanitizing element to reach the valve surface(s) to be sanitized during a sanitizing procedure.

The sanitizing elements of sanitizing articles of the invention also include one or more sanitizing reagents. Sanitizing reagents comprises an active ingredient capable of sanitizing a surface of a needleless medical valve. Any active ingredient, or combination of active ingredients, that can be used effectively to rapidly sanitize a medical fitting or medical line connector (e.g., a needleless medical valve) can be adapted for use in practicing the invention, and are generally classified as antibacterial and antifungal agents, antiseptic or antimicrobial agents, wide spectrum disinfectants, and/or parasiticides, as well as combinations of such reagents. Particularly preferred are biocompatible active ingredients and sanitizing reagents, as the devices of the invention are intended for human and/or veterinary use, including alcohols, antibiotics, oxidizing agents, and metal salts. Sanitizing reagents are preferably in liquid form, with the liquid wetting the substrate. In other embodiments, the substrate is dry and contains a sanitizing reagent dispersed therein. Preferably a sanitizing reagent does not appreciably cross-react with a materials from which needleless medical valve are constructed, and is compatible with the materials used to form the substrate and sealable container of the sanitizing article according to the invention.

In the articles of the invention, the sanitizing element is disposed in a sealed, easily opened container having at least two layers formed from any suitable material, or combination of materials. Representative examples of such containers include pouches and packets. Here, a “pouch” or “packet” refers to a structure made to contain at least two layers, an upper (or first) layer and a lower (or second) layer, joined together (i.e., “sealed”) about their peripheries to form at least one internal cavity adapted to a contain a sanitizing element. The layers may be formed from separate pieces of the same or different material(s); alternatively, they may be formed from the same piece of material such that they can be joined to produce the desired container configuration. For example, a piece of material having the dimensions: length=2X and width=X can be folded about an axis such that upon folding, each of the upper and lower layers have length and width dimensions equal to X. Accordingly, suitable containers can be formed from combinations of separate and/or folded pieces of any suitable material, or combination of materials.

Particularly preferred multi-layered containers are laminated foil pouches or packets formed to have an internal cavity in which the sanitizing element is disposed. Because the sanitizing element is attached to fixedly attached or secured to at least the upper (or first) layer or lower (or second) layer of the container, or to a portion of each of the upper (first) and lower (second) container layers, the sanitizing element is typically affixed to the container layer(s) to which it is attached prior to joining the upper (first) and lower (second) container layers. As those in the art will appreciate, to form such a container, upper (first) and lower (second) layers of substantially the same dimensions (e.g., length and width, curvature, etc.) are aligned and brought in position (i.e., their edges are brought into registration) such that the outer edges of the layers abut one another and the inner surfaces of the upper and lower container layers face one another, facilitating bonding of the two layers about their respective peripheral edges by any suitable technique, such as compression with or without adhesive, heating, welding, etc. Alternatively, the upper and lower layers can be joined without edge registration, after which excess amounts of material can be trimmed away.

Depending upon the particular container configuration and material(s) used, in order to use the device to sterilize a medical fitting, e.g., a needleless medical valve, the container must be opened to expose the sanitizing element. A health care worker can open the container in any suitable way, such as by tearing or cutting it open, peeling apart the first and second layers, etc. although the particular technique used to open the container will typically depend on the container's sealed configuration and the material(s) used to form the upper and lower container layers. When opened, the layers forming the container may be partially or completely separated to expose the sanitizing element contained in the internal cavity of the container when it is sealed, with it being understood that the sanitizing element remains attached to at least one of the layers of the container upon opening and during use. As a result, in use to sanitize a medical fitting it is generally preferred that a health care worker grasp the opened sanitizing device via the outer surface of the container so that the sanitizing element is exposed and can be brought in to sanitizing association with the fitting to be sanitized. “Sanitizing association” means engaging the surface(s) a medical fitting to be sanitized with the sanitizing element of a sanitizing device of the invention, thereby allowing the medical fitting to be sanitized. Because medical fittings such as needleless medical valves often have exposed surfaces with complex external shapes (see, for example, FIG. 11), the article's sanitizing element should be sufficiently compliant and resilient so as to readily conform to the surface features of the needleless medical valve to be sanitized.

In general, the sealed single-use sanitizing articles of the invention are sterile, labeled, and packaged in bulk and provided to health care providers in bulk.

Other aspects of the invention relate to patentable methods of making and using the sanitizing articles of the invention, as well as to patentable methods for reducing a patient's infection risk. Still other aspects concern hand-held machines that use sanitizing articles of the invention to sanitize needleless medical valves.

Other features and advantages of the invention will be apparent from the following drawings, detailed description, and appended claims.

### BRIEF DESCRIPTION OF THE FIGURES

FIG. 1 depicts six different configurations (A-F) for sealed, single-use sanitizing articles according to the invention.

FIG. 2 depicts in four panels (A-D) the opening of a single-use sanitizing article according to the invention having a container that employs a “butterfly” configuration.

FIG. 3 depicts two different configurations (A and B) for sealed, single-use sanitizing articles according to the invention.

FIG. 4 shows side or profile views of three different single-use sanitizing articles according to the invention (panels A, B, and C).

FIG. 5 shows side or profile views of six different single-use sanitizing articles according to the invention (panels A-F).

FIG. 6 shows another embodiment of a sanitizing article according to the invention.

FIG. 7 has two panels, A and B, showing the sanitizing article depicted in FIG. 6 being brought into sanitizing association with a needless medical valve.

FIG. 8 shows a preferred partially folded, just-opened “butterfly” embodiment of a single-use sanitizing article according to the invention.

FIG. 9 illustrates a particularly preferred embodiment of a sanitizing article according to the invention.

FIG. 10 shows a particularly preferred embodiment of a sanitizing article according to the invention in near sanitizing association with a needless medical valve.

FIG. 11 shows two cutaway drawings of a representative diagrammatic illustration of some of the major components of a conventional needleless medical valve having double seal compression points. In panel A, the valve is shown in an open position, with the fluid path indicated by arrows. Panel B shows the valve in a closed position.

FIG. 12 illustrates side or profile views of an additional six representative sanitizing articles according to the invention (panels A-F).

FIG. 13 shows a view from above of each of six additional representative sanitizing articles according to the invention (panels A-F).

FIGS. 14-16 show several alternative embodiments of sanitizing articles according to the invention.

FIG. 17 depicts a sanitizing article embodiment that can be secured over a needleless medical valve after being used to sanitize the valve.

### DETAILED DESCRIPTION

As those in the art will appreciate, the following detailed description describes certain preferred embodiments of the invention in detail, and is thus only representative and does not depict the actual scope of the invention. Before describing the present invention in detail, it is understood that the invention is not limited to the particular aspects and embodiments described, as these may vary. It is also to be understood that the terminology used herein is for the purpose of describing particular embodiments only, and is not intended to limit the scope of the invention defined by the appended claims.

This invention concerns patentable single-use sanitizing articles that can be used to effectively and efficiently clean, disinfect, and preferably sterilize, exposed surfaces of medical line connectors, particularly those of needleless medical valves that at times become part of a fluid communication pathway for introduction of fluids (e.g., IV fluids, blood, plasma, medicines, etc.) into a patient, as these surfaces are at risk for contamination with infectious agents such as bacteria, fungi, and viruses. “Single-use” (or “single purpose”) refers to an article or device suitable for one use or purpose only, as distinguished from “dual” or “multiple” use or purpose devices. Thus, in the context of the invention, a “single-use” sanitizing article or device is one that is useful for sanitizing, for example, a needleless medical valve or, at least with respect to some embodiments, a region of skin of a subject. After such use, the device is no longer suitable for any further use or purpose and is to be discarded. In contrast, a dual-use device would include one suitable for both sanitizing a medical fitting and then serving as a cap to minimize exposure of the valve to infectious agents when the valve is not being used to provide access to the patient's vasculature.

In general, the single-use sanitizing articles of the invention each comprise a sanitizing element disposed in an easily opened multi-layered container such that the sanitizing element can be maintained in a clean, preferably sterile, condition until it is used to sanitize (i.e., clean, disinfect, or sterilize) a medical line connector or fitting such as a needleless medical valve (sometimes also referred to a “luer access device”). The sanitizing element is configured such that, when brought into sanitizing association with, for example, a luer access device, it readily conforms to the three dimensional external structure of that portion of the luer access device that one desires to sanitize in order to sanitize the accessible, and potentially contaminated, surfaces of the luer access device.

Herein, a sanitizing element comprises a sanitizing reagent dispersed in a substrate. In some embodiments, the sanitizing reagent is dispersed in or otherwise combined with the substrate during the process used to manufacture the sanitizing device, while in other embodiments, the device is configured such that the sanitizing reagent is released for dispersion into the substrate post-manufacture, but prior to the time the device is brought into contact with the needleless valve to be sanitized.

In accordance with the invention, a sanitizing reagent comprises an active ingredient capable of sanitizing a surface of a needleless medical valve. Any active ingredient that can be used effectively to rapidly sanitize a medical fitting or medical line connector (e.g., a needleless medical valve) can be adapted for use in practicing the invention, and are generally classified as antibacterial and antifungal agents, antiseptic or antimicrobial agents, wide spectrum disinfectants, and/or parasiticides, as well as combinations of such reagents. Particularly preferred are biocompatible active ingredients and sanitizing reagents, as the devices of the invention are intended for human and/or veterinary use, including alcohols, antibiotics, oxidizing agents, and metal salts. Representative examples of such active ingredients include bleach, chlorhexidine, ethanol, isopropyl alcohol, hydrogen peroxide, sodium hydroxide, and an iodophor dissolved or otherwise dispersed in a suitable solution, suspension, or emulsion. Other active ingredients having suitable sanitizing effects can also be used. These include alcohols (e.g., ethanol, benzyl alcohol, isopropyl alcohol, phenoxyethanol, phenethyl alcohol, etc.); antibiotics (e.g., aminoglycosides, such as amikacin, apramycin, gentamicin, kanamycin, neomycin, netilmicin, paromomycin, rhodostreptomycin, streptomycin, and tobramycin; bacitracin; chloramphenicol; erythromycin; minocycline/rifampin; tetracycline; quinolones such as oxolinic acid, norfloxacin, nalidixic acid, pefloxacin, enoxacin and ciprofloxacin; penicillins such as oxacillin and pipracil; nonoxynol 9; fusidic acid; cephalosporins; etc.), quaternary ammonium chlorides; quaternary ammonium carbonates; benzalkonium chloride; chlorinated phenols; fatty acid monoesters of glycerin and propylene glycol; iodine; iodine containing compounds, such as 3-iodo-2-propynyl butyl carbamate (IPBC); iodophors, such as povidone-iodine (Betadine 100%, which contains providine iodine as the active ingredient); hydantoins, such as dimethylhydantoin and halogenated hydantoins; isothiazolinones; parabens, such as methylparaben, ethylparaben, and propylparaben; chloroxylenol; chlorhexidine and its salts; chlorhexidine/silver-sulfadiazine; chlorhexidine acetate; chlorhexidine gluconate (e.g., Hibiclens); chlorhexidine hydrochloride; chlorhexidine sulfate; benzoic acid and salts thereof; benzalkonium chloride; benzethonium chloride; methylbenzethonium chloride; chlorobutanol; sorbic acid and salts thereof; imidazole antifungals (e.g., miconazole); butocouazole nitrate; mafenide acetate; nitrofurazone; nitromersol; triclocarban; phenylmercuric nitrate or acetate (0.002%); chlorocresol; chlorbutol; clindamycin; CAE (Anjinomoto Co., Inc., containing DL-pyrrolidone carboxylic acid salt of L-cocoyl arginine ethyl ester); cetylpyridinium chloride (CPC) at 0.2%, 0.02%, and 0.002% concentrations; 9.8% isopropyl alcohol; 1% ZnEDTA; mupirocin; and polymyxin (polymyxin b sulfate-bacitracin). Additionally, other useful compounds and compositions include Miconazole, Econazole, Ketoconazole, Oxiconizole, Haloprogin, Clotrimazole, butenafine HCl, Naftifine, Rifampicin, Terbinafine, Ciclopirox, Tolnaftate, Lindane, Lamisil, Fluconazole, Amphotericin B, Ciprofloxecin, Octenidine, Triclosan (2,4,4′-trichloro-2′-hydroxydiphenyl ether), Microban (5-chloro-2-phenol (2,4 dichlorophenoxy). Useful metals include silver and its salts, including silver acetate, silver benzoate, silver carbonate, silver citrate, silver iodate, silver iodide, silver lactate, silver laurate, silver nitrate, silver oxide, silver palmitate, silver protein, and silver sulfadiazine. Sanitizing reagents are often compositions that comprise the desired active ingredient(s) in admixture with other ingredients, such as carriers and liquid solvents.

The particular active ingredient(s) selected as a sanitizing reagent for a given application will be compatible with the sanitizing element substrate and material(s) used to form the layers of the container the particular device. In some embodiments, the sanitizing reagent is dispersed in the substrate after the substrate is formed. For example, a sanitizing reagent can be dispersed by saturating or supersaturating a substrate during manufacture of the device, preferably before it is sealed. In other embodiments, the sanitizing reagent can be dispersed during the process used to manufacture the substrate. As will be appreciated, the materials used to prepare the sanitizing reagent should be compatible with the constituent or constituents that comprise the substrate such that the substrate does not appreciably degrade or otherwise suffer loss of structural integrity prior to being used to sanitize a medical valve or region of a patient's skin. Similarly, the sanitizing reagent should be biocompatible, such that it will not harm a patient in the event of contact or should some amount of the sanitizing reagent be admitted into the fluid carrying portion of a needleless medical valve, as well as with materials used to form needleless medical valves.

In preferred embodiments, the substrate used to form a sanitizing element is any suitable absorbent, compliant, pliable, resilient, fibrous or porous material, or combination of materials, than can be wetted and/or impregnated with a sanitizing reagent and which can easily and readily adapt to complex surface contours (e.g., luer threads, concave and convex surfaces, flanges, etc.) likely to be engaged upon contact with a needleless medical valve to be sanitized. Such materials include those that are synthetic or naturally occurring, and they may be of homogeneous or heterogeneous composition. Preferred synthetic materials include fibrous, foam, and gel compositions, particularly those having directionally oriented natural or synthetic fibers, or combinations thereof. Preferred naturally occurring materials useful as substrates include fibrous naturally occurring materials, including plant-derived materials such as cotton and paper products, as well as animal-based fiber products such as wool. Other preferred natural materials are sponges.

As will be appreciated, in order to achieve the desired sanitizing effect, a sanitizing element, or the component part(s) thereof designed to contact a medical fitting such as a needleless medical valve, preferably are made of a material (or combination of materials) that allow the sanitizing element to thoroughly sanitize surfaces of medical fittings such as needleless valves or luer access devices, particularly those surfaces that are exposed to air and thus are at risk for contamination with infectious or pathogenic agents, and are also intended to form part of the fluid flow path for fluids to be introduced into a patient, for example, IV solutions, medications, blood and blood products, etc.

Preferably, the substrate material and container layer(s) to which the substrate is secured should be sufficiently compliant to allow the sanitizing element and container layer(s) to be deformed using gentle manual pressure in order to conform to the external structures present on the surface(s) of the medical fitting to be sanitized. This assures intimate, sanitizing contact between the sanitizing and at least those exposed surfaces of, for instance, a needleless medical valve designed to come into contact with fluid entering the valve, such as IV fluids. In addition, the substrate allows for the retention of a liquid sanitizing reagent, for example, in capillary spaces, in the void volume of sponges, etc. The substrate may also be engineered such that its surface is modified to include sanitizing reagents such as silver ions and/or other suitable materials.

Preferred natural materials include those derived from cotton and naturally occurring sponges. As those in the art appreciate, processed cotton fibers are composed almost entirely of the natural polymer cellulose. In such fibers, 20-30 layers of cellulose are coiled into a series of spring configurations, which makes the fibers absorbent and gives them a high degree of durability and strength. For example, woven cotton sheets, as are often used in the manufacture of sterile cleansing pads that are then saturated with a 70% isopropyl alcohol (IPA) solution, can be used as substrates for sanitizing elements according to the invention. Any suitable configuration may be used. For example, a woven cotton sheet can be cut into numerous similarly sized pieces, each of which can be used as a substrate. In many embodiments, after attachment to the inside surface of a layer of the container (e.g., through the use of an adhesive, double-sided, tape, etc.), the substrate is ready for the addition of a suitable sanitizing reagent. Alternatively, cotton fibers can be spun onto the inside surface of a layer of the container. Other fibers, be they naturally occurring, synthetic, or combinations of natural and synthetic materials, having similar properties can also readily be adapted for use as substrates to make sanitizing elements.

Another class of materials for substrate fabrication is directionally oriented fibrous materials. These include, without limitation, materials comprised of cellulose fibers, glass fibers, and polyester fibers, as well as materials comprised of combinations of two of more of these and/or other materials. Such bonded synthetic fibers use capillary action to precisely absorb, retain, transfer, and/or release liquids or vapor in desired amounts. A broad range of synthetic polymers can be used to form the fibers, and, if desired, they may be treated for functional purposes, for example, to contain a sanitizing reagent dispersed therein, to provide a vapor barrier or other coating over a portion of the product's surface, etc. The geometric shape of these materials can also be customized for particular applications, thereby permitting easy integration into substrate configurations having the desired device thickness, widths, length, diameter, etc.

Other representative classes of materials suitable for use as substrates include gel-forming polymers and foams such as agarose, agar, polyacrylamide, and other synthetic porous materials that can be formed into layers, sheets, columns, or other shapes compatible with practicing the invention. Representative gelatinous materials include hydrogels (i.e., cross-linked polymers that absorb and hold water), particularly those made from agarose, (2-hydroxyethyl)methacrylate and its derivatives, and synthetic carbohydrate acrylamides.

Still other classes of materials include porous polymer sponges. Such sponges can be formed from any suitable material, including polyethylene, polypropylene, olytetrafluoroethylene, polyvinylidine difluoride, polynitrile, and polystyrene. Many such porous polymer sponges are commercially available in a wide variety of shapes, pore density and size, etc. Additionally, polymer sponges can be made by polymerizing appropriate monomers according to conventional foam forming techniques. In general, sponges have an open pore structure to allow movement of a solvent such as a liquid sanitizing reagent. The sponge surface should include open pores to provide entry of liquid sanitizing reagents (e.g., alcohol, iodine-containing solutions, etc.), and, as with other materials used to form substrates, the particular substrate material chosen is preferably inert, i.e., not reactive with components of the sanitizing reagent, the shell of the article or its container, or the materials used to produce medical fittings such as needleless medical valves.

Surgical foams are another preferred class of substrate materials. The materials can be natural or synthetic, as desired. Suitable foams include rubber latex, polyurethane, polyethylene and vinyl foams. Preferably, such foams are made from any suitable biocompatible polymer, for example, polyvinyl alcohol (PVA) or polyurethane. One preferred foam material is Microbisan™, a hydrophilic polyurethane foam that is impregnated with silver ions (Lendell Manufacturing, St. Charles, Mich.). Preferably, such foams are highly absorbent and thus suitable for use with liquid sanitizing reagents. In other embodiments, the material used to form the foam is well-suited for dispersion of a dry sanitizing reagent, such as silver ions. Again, it is preferred that foam materials, if used to as a substrate, be inert. Also, they are preferably sufficiently flexible to conform to the variety of different shapes and surface configurations (e.g., double seal fluid access points, luer threads, etc.) encountered in the field given the multitude of medical valve shapes, sizes, and configurations. In this way sufficient contact between the sanitizing surface(s) of the sanitizing element and the surface(s) of the medical valve to be cleansed can be ensured. Another advantage of some synthetic foams (as well as certain other polymeric materials from which substrates may be formed) is that they can easily be injected in a desired volume into a shell or housing during manufacture, after which they expand to assume the desired substrate size, density, porosity, etc.

Furthermore, sanitizing elements can include chemicals to indicate a functional change in the substrate, for example, by using a color change to signal a change from a wet to a dry state, or, alternatively, that the substrate has been properly wetted with a liquid sanitizing reagent dispersed into the substrate by a health care worker just prior to use, as opposed to during manufacture of the device. Thus, depending on the system used, a color change in the substrate could be used to indicate that the substrate has dried out and should not be used, perhaps due to a leak in the article's storage container. Alternatively, when, for example, a colored liquid sanitizing reagent is used, the user can visually confirm dispersion of the reagent in the substrate by assessing whether the colored sanitizing reagent is dispersed throughout the substrate.

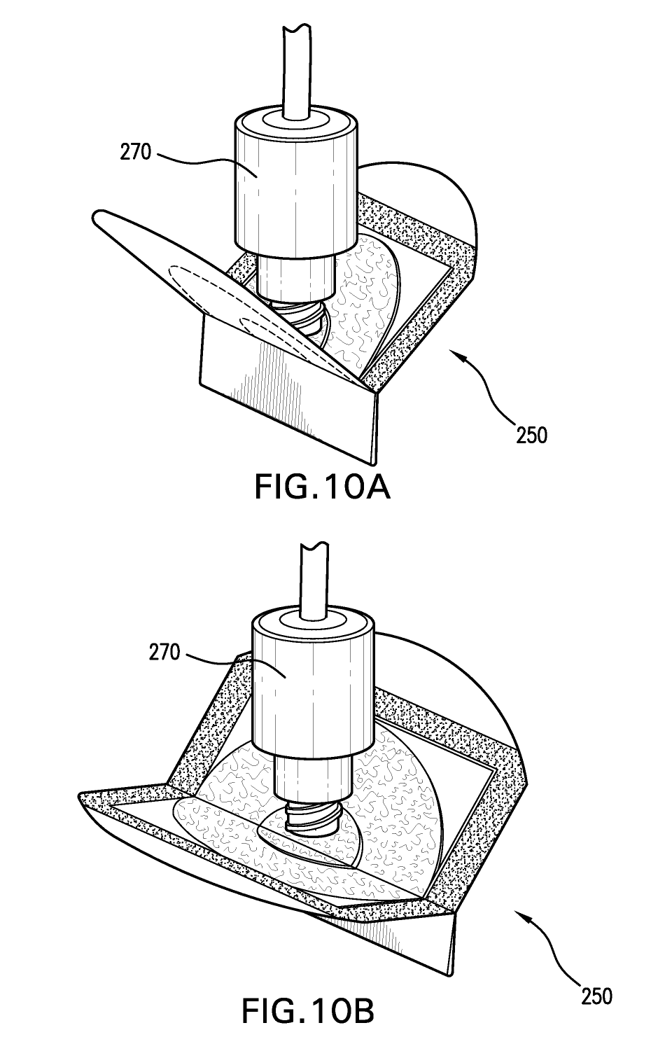

The sanitizing element of a device according the invention preferably includes a readily apparent sanitizing region designed to engage an accessible surface of a valve stem of a needleless medical valve so as to expose the externally accessible valve surface, and any infectious agents residing thereon, to the sanitizing reagent(s) carried in the substrate. In many embodiments, the sanitizing region is the centrally exposed, accessible outward-facing surface (i.e., a sanitizing surface) of the sanitizing element designed to contact the surface(s) of the medical fitting to be sanitized. For example, the sanitizing region can be a built up or thicker central region of a sanitizing element sized to have a diameter substantially the same as the distal end of a needless medical valve (inside which the depressible valve stem of the valve is located, the depression of which provides access to the fluid path inside the body of the valve; see, e.g., FIG. 10). In some embodiments, the portion of layer(s) of the substrate and/or container that underlies a sanitizing region is structurally reinforced to ensure that the sanitizing surface of the sanitizing region does not appreciably deform during a sanitizing procedure.

In some embodiments, an abrasive layer may be disposed on or comprises the upper surface of the substrate or portion thereof, such as to form an abrasive layer on the upper surface of a sanitizing region of a sanitizing element. An abrasive layer typically is comprised of a natural or synthetic material, or combination of materials, that provide it with a greater abrasive or scrubbing capacity than material used to form other portions of the substrate, thereby enabling the abrasive layer to provide greater capacity to assist in the mechanical disruption or removal of biofilms (as, for example, may be formed by infectious agents contaminating the exposed surface(s) of needleless medical valves in a PIVC or CVC connected to a patient in a hospital or other healthcare setting) or other unwanted materials. It will be understood that an “abrasive layer” can be formed in the upper portion of the substrate that includes the sanitizing region by a suitable treatment, such as heating, chemical treatment, and the like.

As already described, in some embodiments, the sanitizing element comprises a single layer (which layer may be of substantially uniform thickness, or, alternatively, may have different thicknesses), whereas in others, it comprises a plurality of layers. In multi-layer devices, the substrate used to form each layer can be of the same or different material, and may or may not contain a sanitizing reagent. Additionally, in some embodiments of multi-layer devices, one or more of the layers may be physically separated from the other substrate layer(s) it contacts by an impermeable, semi-permeable, or permeable barrier.

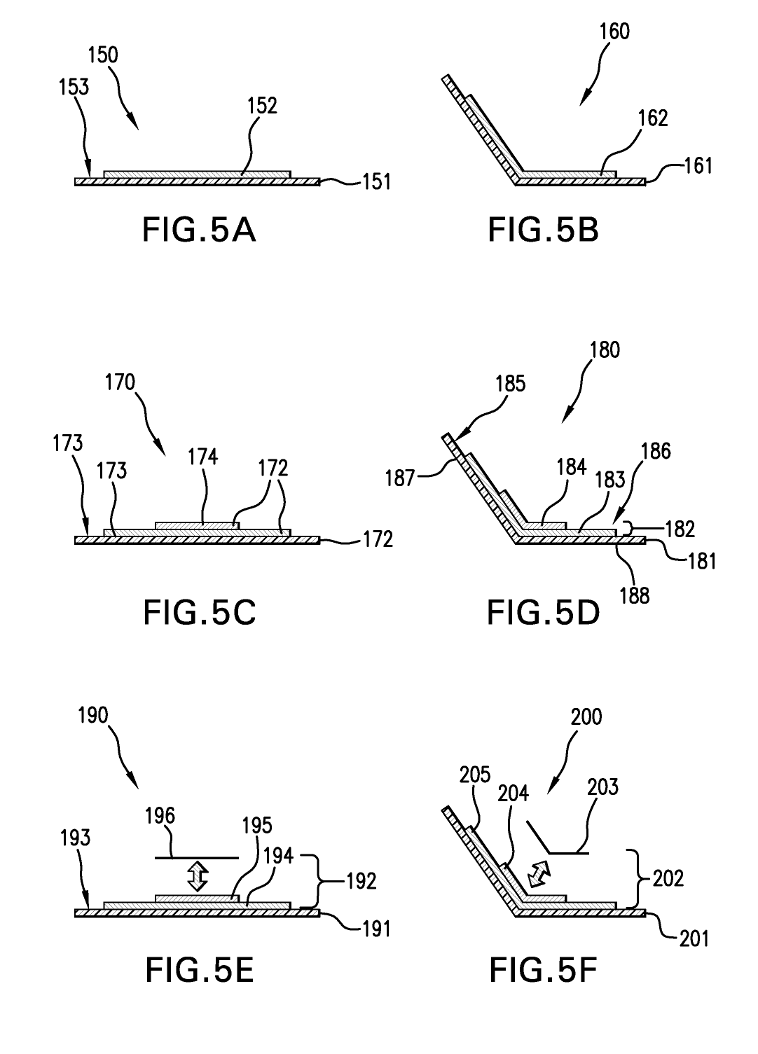

In other embodiments, a substrate varies in thickness (when viewed, for example, in cross-section; see FIG. 5, for example). This can be accomplished, for example, through the use of two or more stacked layers. Alternatively, thickness can be varied by depositing different amounts of the same material(s) at different locations on the surface that will be the inner surface of the piece or portion of material used to form that part of the container to which the substrate is to be attached. Alternatively, the substrate can be disposed on a backing material suitable for fixed attachment to the piece or portion of material used to form that part of the container to which the substrate is to be attached.

For sanitizing elements that comprise multi-layered substrates, at least one of the layers contains a sanitizing reagent. In some such embodiments, each layer contains the same or a different sanitizing reagent. Here, a “different sanitizing reagent” means that each reagent contains either a different active ingredient(s), or the same active ingredient(s) in a different formulation or concentration. When different active ingredients are used, they are preferably compatible, such that one does not inactivate or otherwise degrade the sanitizing activity of the other active ingredient(s), nor should it materially degrade or chemically alter any substrate used to form a substrate layer or any material used to manufacture a medical fitting that can be sanitized by the device of the invention.

In embodiments wherein the sanitizing element is comprised of two or more layers, the substrate portion of each layer can be formed from a material that is the same as or different from the material used to form the substrate of one or more of the other layers, and each layer may contain the same, different, or even no, sanitizing reagent (although at least one layer will have a sanitizing reagent dispersed therein prior to engaging the surface of the needleless valve to be sanitized). Also, even when substrates for different layers are formed from the same material, they may be configured differently. For example, in a particularly preferred embodiment that employs a sanitizing element having two layers, where the substrate for each layer is formed from the same type of natural absorbent material, such as woven cotton, the lower layer (which is attached to the inner surface of a layer of the container) is larger, whereas the second layer is smaller and positioned in the center of the larger lower cotton layer.

In any single-use sanitizing article according to the invention, prior to use the sanitizing element (or substrate prior to addition of the sanitizing reagent) is encapsulated, enclosed, or housed in a container having at least two layers. When the container is opened, for example, by peeling apart the first and second layers of the container, the sanitizing element is exposed so that it can be brought into sanitizing association with the surface to be sanitized, for example, an accessible surface of a valve stem of a needleless valve of a medical fitting. The sanitizing element is fixedly attached or secured to at least one layer of the container. The attachment between the sanitizing element and container layer(s) is permanent, and can be accomplished using any suitable chemical, mechanical, or other attachment technology. As will be appreciated, a device according to the invention can also include a gripping element or region to facilitate imparting twisting, rotating, and/or plunging action on a sanitizing article while it engages a medical fitting, such as a needleless medical valve to be sanitized prior to connection to a reservoir (e.g., an IV bag or syringe) for delivery of a solution to a patient.

In some embodiments where a container has a single internal cavity once it is sealed, multiple components can be included therein. For example, a substrate and a reservoir containing a liquid sanitizing reagent can reside within the cavity upon sealing. In other embodiments the container is formed to have a plurality of cavities or internal spaces. For example, one cavity can contain a sanitizing element substrate, whereas another cavity can contain a liquid sanitizing reagent. In such embodiments the cavities can be separated by, for example, an impermeable layer that can be ruptured to release the liquid sanitizing reagent so that it can wet the substrate.

For example, a container can contain a sealed fluid reservoir or fluid chamber charged with a volume of sanitizing reagent. Fluid containing the sanitizing reagent can then be released from the reservoir or chamber just prior to using the device. For example, in one such embodiment the device includes a previously manufactured packet containing a desired volume of a desired sanitizing reagent. The fluid-filled packet is placed in the container during manufacture prior to the container being sealed. Just prior to use the packet can be ruptured, for example, by squeezing the pouch to release its contents. The liquid sanitizing reagent is then absorbed into the substrate of the sanitizing element. In a preferred embodiment of this type, such wetting of the substrate results in a color change of the sanitizing element that can be perceived by a health care worker once the container is opened. The health care worker will then know, by virtue of the color of the sanitizing element, that the device is suitable for use to clean a needleless medical valve. As a representative example, the liquid sanitizing reagent stored in the packet can be manufactured to include a biocompatible dye or coloring reagent (for example, a blue dye). When the packet is ruptured and the blue liquid sanitizing reagent is, for example, absorbed by the substrate, for example, white woven cotton, the substrate appears to change color, from white to blue. Thus, the health care worker will know that if the sanitizing element is blue, it is ready to use; however, if it is not blue, or is only blue in certain areas (e.g., because the packet containing the liquid sanitizing reagent did not rupture, the liquid sanitizing reagent did not completely wet the substrate after being released from its packet, the integrity of the pouch was comprised to an extent that some or all of the substrate was damaged and unable to absorb the liquid sanitizing reagent after its release from the packet, etc.), s/he will know not to use it.

Sanitizing articles of the invention can be made using any suitable process adapted for use with the materials used to form the sanitizing element and container. Particularly preferred are laminated foils. Automated form, fill, and seal machines can be adapted for such uses.

Preferably, the outer surface of the container intended for grasping by a user has a non-slip surface, i.e., one having a high coefficient of friction so that when the sanitizing article is held in a user's hand and positioned to sanitize a medical fitting, it can be manipulated, for example, using a twisting or rotating motion, with minimal or no slippage in the user's bare or gloved hand. Examples of such surfaces include those having ridges, valleys, dimples, bumps, or other features designed to enhance friction, as well as combinations of two or more of such features. Such features can be introduced into the container's outer surface as part of the manufacturing process, and if desired in a particular application, materials having high grip levels can also be used to fabricate the one or more of the layers of the container. Alternatively, a non-slip coating can be applied to one or more of the outer surfaces (e.g., the upper and/or lower surfaces) of the container.

In general, the sanitizing articles of the invention are provided to users in a sealed, sterile manner. If desired, labeling information, logos, artwork, manufacturing, and/or regulatory data (e.g., lot number, expiration or “use by” dates, etc.) may also be printed or otherwise applied to individual sanitizing articles. In addition, information such as a bar code (to allow use of the device to tracked, for example) may also be included on individual sanitizing articles. In particularly preferred embodiments, packaged sanitizing articles are sterilized using a suitable process, such as irradiation. As will be appreciated, sanitizing articles may be packaged individually or in groups of two or more units as kits, which can further include instructions for use of the sanitizing article(s).

In a particularly preferred practice, the sanitizing articles of the invention are sterilized as part of the manufacturing process. Here, “sterilization” refers to any process that effectively kills or eliminates transmissible agents, e.g., bacteria, viruses, fungi, prions, spores, etc. that may be present in any component of a device according to the invention. In preferred embodiments, sterilization can be achieved by heating, chemical treatment, irradiation, and other processes. Indeed, any sterilization process compatible with the materials used to make the sanitizing device can be employed. A particularly preferred sterilization process is an irradiation process. Such processes include irradiation with x-rays, gamma rays, or subatomic particles (e.g., an electron beam). In general, when a sterilization process is used in the context of the invention, the process is employed on a sanitizing article after it has been sealed and/or packaged.

The invention also concerns methods of using the instant single-use sanitizing articles of the invention. Such methods include using the articles to sanitize medical fittings such as needleless medical valves, luer access devices, and the like. To perform such methods, the sanitizing element of a single-use sanitizing article is contacted with (i.e., brought into sanitizing association with) the surface of the medical fitting to be sanitized, typically just before it is to be connected to a fluid delivery device (e.g., syringe) or fluid-containing medical reservoir (e.g., an IV bag, blood or blood product storage bag, etc.) that contains a solution to be delivered to a patient. In preferred practice, once the sanitizing region is in contact with the surface(s) of the medical fitting to be sanitized, the article is moved in relation to the fitting, for example, by rotation or twisting. Such contact and sanitizing action can be for any desired period, with periods of about one second to about ten to twenty seconds being particularly preferred. After contact, the article is removed from the medical fitting and discarded, after which, for example, a fluid-containing medical reservoir is immediately connected to the now-sanitized fitting. In preferred embodiments where the sanitizing reagent is a solution, the surface(s) of the fitting contacted with the sanitizing element are allowed to dry (or are dried, for example, by wiping with a sterile, absorbent cloth or wipe, which cloth or wipe may be dry or wetted with a volatile, compatible solution such as 70-100% alcohol) prior to connecting the fitting to a fluid reservoir. In preferred practice such sanitizing methods result in at least a 2-fold, 5-fold, or 10-fold or more reduction in microorganism contamination on the accessible surface(s) that have been sanitized, Even more preferably, the level of reduction may exceed a 100-fold, a 10³-fold, a 10⁴-fold, a 10⁵-fold, a 10⁶-fold, or 10⁷-fold reduction in microorganism contamination on the accessible fitting surface.

In addition to methods for sanitizing accessible surfaces of luer access devices and the like, the articles of the invention provide methods of reducing infection risk in a patient connected to devices, such as a peripheral IV line, a central IV line, or a peripherally inserted central catheter, configured for delivering fluids directly into the patient's blood stream. The risk reduction afforded by the articles of the invention may vary depending upon many factors, such as patient age and condition, the condition being treated, the location where medical services are being delivered, patient density, the level of contaminating microorganisms in the environment, the quality of air handling equipment in the medical facility, the degree of training of medical personnel charged with sanitizing the access device, the method(s) used to periodically sanitize the medical fitting, interval between sanitizing procedures, the particular configuration of the sanitizing article, the particular configuration of the medical valve, etc. Risk reduction can be established using any suitable method, for example, by assessing HAI frequency in the presence and absence of using sanitizing articles according to the invention. Reductions of HAI infection risk of 1-100% or more, including up to 1000% or more, are envisioned. As will be appreciated, reductions in infection risk (e.g., HAI risk) will translate to improved patient outcomes (through reduced morbidity and mortality) and reduced expenditure on treating HAI's.

It will be appreciated that the articles of the invention can be used manually.

### Representative Embodiments

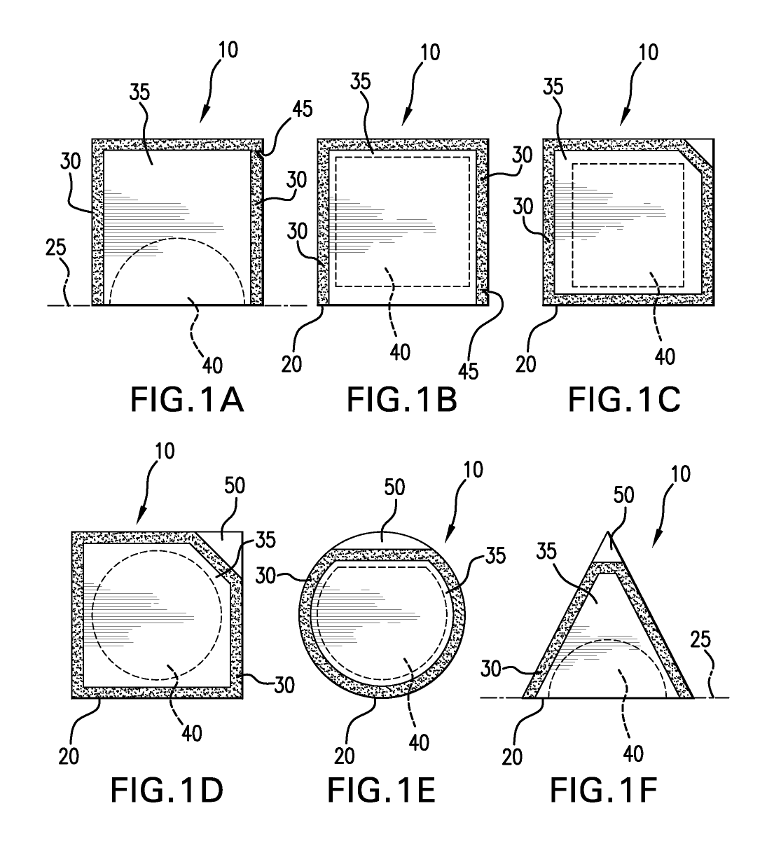

The following descriptions concern several representative embodiments of the invention, which are illustrated in FIGS. 1-10.

FIG. 1 depicts six different configurations of a sealed, single-use sanitizing article (10) according to the invention, labeled A-F. In each configuration a container (20) is made of 2 layers, a first layer and second layer (which layer are not distinguished in A-F). In the embodiments shown in panels A and F, the first and second layers are made from the same piece of material (e.g., a laminated foil) and are folded about an axis (25). In the embodiments shown in panels B-E, the first and second layers are separate pieces, and can be of the same or different material. The first and second layers are sealed about their peripheral edges (30; spotted regions) to form an internal cavity (35). In each configuration the boundary of the sanitizing element (40) sealed within each container, and attached to either (B-E) or both (A and F) of the first and second layers, is indicated by a hatched line. The containers shown in panels A and B have a notch (45) placed in one of the sealed edges to facilitate opening (by tearing) of the container. The containers shown in panels C—F have a “butterfly” (50) configuration to facilitate opening of the container by peeling the first and second layers apart.

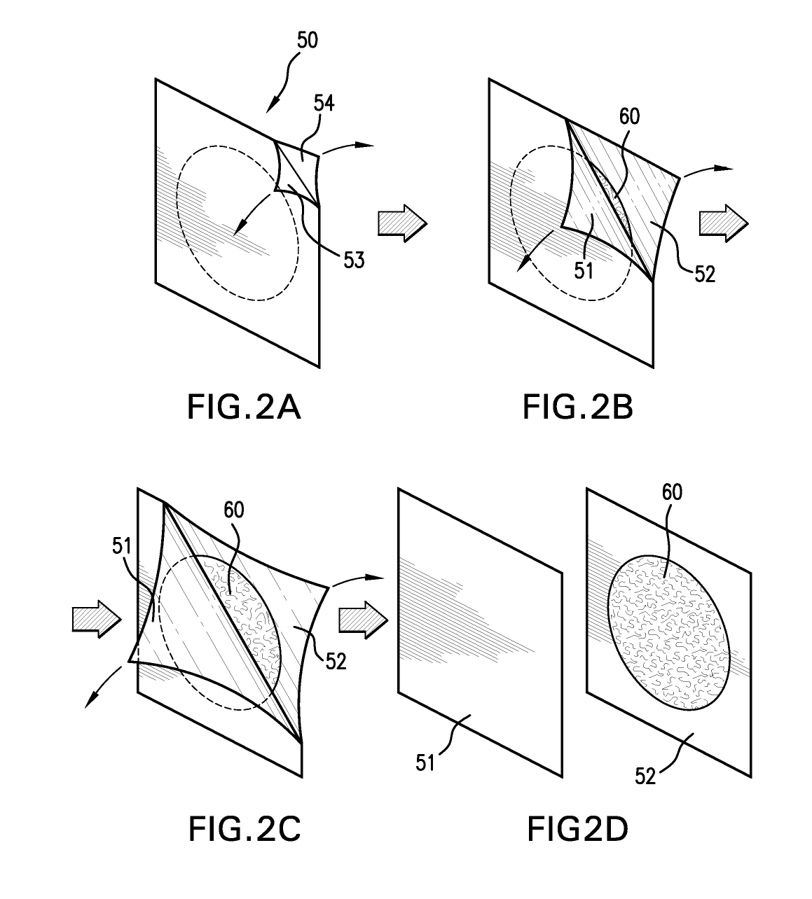

FIG. 2 depicts the opening of a sealed, single-use sanitizing article (50) that uses a two piece (51, 52) “butterfly” container as illustrated in the FIG. 1D. Each piece, or layer (51, 52), of the container has at one of its corners a free “ear” (53, 54) that can easily be separated from the “ear” of the other layer of the container so that the container can be peeled open to expose the sanitizing element (60) securely attached to the one of the layers (52) of the container. FIG. 2 has four panels, A-D, that illustrate the opening of the container (50). Initially, the free ears (53, 54) of each layer (51, 52) are spread apart and then pulled (small arrows), which begins to reveal a portion of the sanitizing element (60). As the container layers (51, 52) are separated from each by pulling on the free ears (53, 54), more of the sanitizing element (60) is exposed (panel C). Finally, when the two layers (51, 52) of the container are completely separated (panel D), the sanitizing element (60) is completely exposed and ready to use to sanitize a medical fitting such as a needless medical valve (not shown).

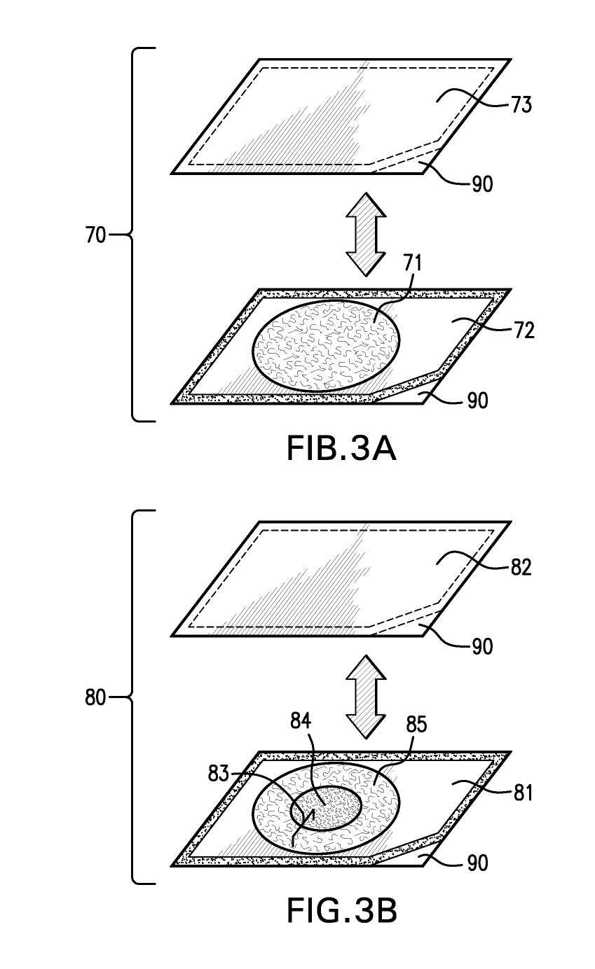

FIG. 3 depicts two alternative embodiments (panels A and B) of a sealed, single-use sanitizing article (70, 80) according to the invention. In each embodiment, the container is two pieces, or layers, that are separated from each other prior to using the article. In the embodiment shown in FIG. 3A, the sanitizing element (71) is securely attached to the first or bottom layer (72) of the container. The sanitizing element has a substrate made from a single layer, such as woven or spun cotton, to which a sanitizing reagent is then added during manufacture. Thereafter, the second or upper layer (73) to the joined to the bottom layer (72) using a process compatible with the material(s) used for form the upper and lower layers of the container. FIG. 3B shows another sanitizing element (80) embodiment wherein the container is also made from two pieces or layers, and upper layer (81) and a lower layer (82). In this device the sanitizing element (83) comprises two layers (84, 85; or a single layer having two thicknesses). In each of the sanitizing articles shown in panels A and B of FIG. 3, the lower and upper layers (72, 73 in FIG. 3A; 81, 82 in FIG. 3B) are joined or sealed about their respective peripheries (hatched regions about the peripheries of the lower layers in FIGS. 3A and 3B; dotted regions about the peripheries of the upper layers in FIGS. 3A and 3B), with a free “ear” (90) being left at one corner of each of the upper and lower layers in each container.

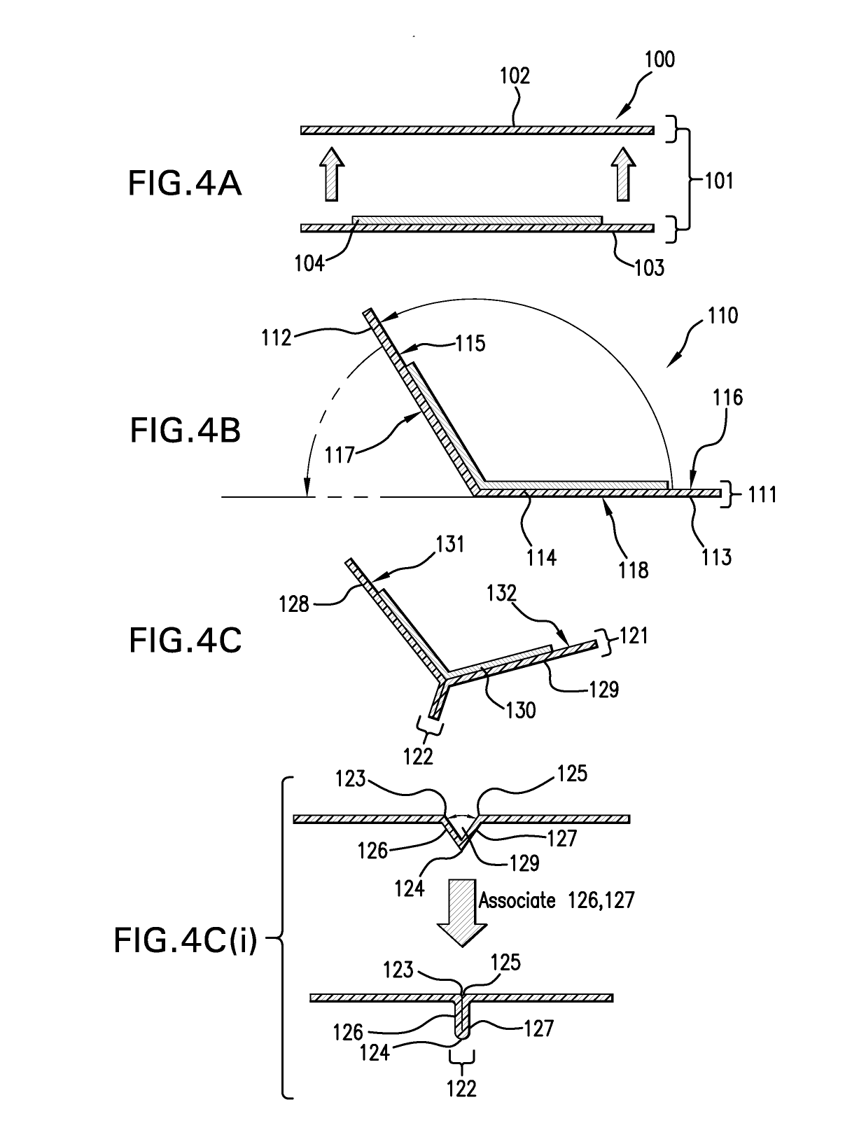

FIG. 4 depicts three alternative embodiments (panels A-C) of a sealed, single-use sanitizing article (100, 110, 120) according to the invention. FIG. 4A shows a sanitizing article (100) of the sort depicted in FIG. 1D and FIG. 2, wherein the article's container (101) is made from two pieces (an upper layer (102) and a lower layer (103)) that are joined during manufacture and which are separated just prior to use. The sanitizing element is a comprised of a single substrate layer (104) wetted with a liquid sanitizing reagent (indicated by hatching). FIG. 4B illustrates a sanitizing article (110) wherein the container (111) is formed from a single piece of material (e.g., a laminated foil) that is folded to form a first or upper layer (112) connected along one edge to a second or lower layer (113). The sanitizing element is a comprised of a single substrate layer (114) wetted with a liquid sanitizing reagent (indicated by hatching). The sanitizing layer is fixedly attached to the inner surfaces (115, 116) of the first and second layers (112, 113). The outer surfaces (117, 118) of the first and second layers (112, 113) can contain labeling and/or other information (not shown). To use such a sanitizing article, the container is opened, for example, by tearing along off the joined edges of the container opposite the fold and then pulling apart (indicated by the curved arrows) the first and second layers (112, 113) to expose the sanitizing element (114). The user then grasps the sanitizing article such that her/his fingers contact the outer surfaces (117, 118) of the first and second layers (112, 113). The sanitizing article so grasped is then brought into sanitizing association with the medical fitting (e.g., a needless medical valve) to be sanitized.

FIG. 4C is a profile view of another embodiment of a sanitizing article according to the invention. Here, the sanitizing article (120) also comprises a container (121) formed from a single piece of material (e.g., a laminated foil). Here, the container can be formed by making three parallel, equally spaced folds (123, 124, 125; see inset (i)), the first of which is used to produce a grasping element (122) through association of grasping regions (126, 127) of the first (upper) and second (lower) layers (128, 129). Associating the grasping regions (126, 127) of the first and second layers (128, 129) brings the edges formed along the outer folds (123, 125) together. If desired, a stiffening element (not shown) can be inserted in the channel (129) between the grasping regions (126, 127) of the first and second layers (128, 129) before they are brought together. After forming the grasping element the substrate for the sanitizing element (130) is securely attached to the inner surfaces (131, 132) of the first and second layers (128, 129). In this embodiment the sanitizing element (130) is a comprised of a single substrate layer wetted with a liquid sanitizing reagent (indicated by hatching). The outer surfaces (133, 134) of the first and second layers (128, 129) can contain labeling and/or other information (not shown), such as instructions for use, bar coding, etc. To use such a sanitizing article (120), the container (121) is opened, for example, by tearing along off the joined edges of the container opposite the now-adjoining folds (123, 125) and then pulling apart (indicated by the curved arrows) the first and second layers (128, 129) to expose the sanitizing element (130). The user then grasps the sanitizing article by the integral grasping element (122) of the container. The grasped sanitizing article can then be brought into sanitizing association with, for example, a needless medical valve.

FIG. 5 shows six different representative embodiments (A-F; 150, 160, 170, 180, 190, 200) of sanitizing articles according to the invention. In this figure three of the embodiments (A, C, E; 150, 170, 180) represent sanitizing articles wherein the container comprises two pieces, an upper (first) piece (not shown) and a lower (second) piece (151, 171, 191) to which a sanitizing element (152, 172, 192) is secured to the inner surface (153, 173, 193) of the lower piece (151, 171, 191). These embodiments (A, C, E; 150, 170, 180) differ in that the sanitizing elements (152, 172, 192) are comprised of 1, 2, and 3 layers. In FIG. 5C, the sanitizing element (172) has two layers (173, 174). The bottom layer (173) is secured to the inner surface (173) of the lower container piece (171). The top layer (174) of the sanitizing element is secured to the bottom layer (173); alternatively, the top and bottom layers need not be physically distinct elements, but can instead simply be a single substrate element having regions of different thickness. Preferably, the center region (or top layer) of the sanitizing element (172) is sized to engage the distal end of a needleless medical valve. In FIG. 5E, the sanitizing element (192) has three layers (194, 195, 196). In this view of this embodiment, the top-most layer (196) is a thin, abrasive element shown separated from the two layers (194, 195) below it merely for ease of description; in an actual physical embodiment the abrasive layer (196) would be secured to the central section of the sanitizing element positioned below it.

Panels B, D, and F of FIG. 5 show sanitizing article embodiments wherein the multiple layers comprising each container (161, 181, 201) are formed from one piece of material, typically by folding. In FIG. 5B, the sanitizing element (162) is comprised of one layer of substantially uniform thickness. In FIG. 5D, the sanitizing element (182) is comprised of two layers (183, 184) (or a single layer of differing thickness when viewed in cross section), with the top layer (184) secured to and centrally positioned atop the bottom layer (183), which is fixedly attached to the regions of the inner surfaces (185, 186) of the first and second layers (187, 188) of the container (181). In FIG. 5F, the sanitizing element (202) is much the same as in the embodiment shown in FIG. 5D, except that an additional thin abrasive layer (203) is securely attached on top of the absorbent substrate material used to form the lower two layers (204, 205) (or a single layer of differing thickness when viewed in cross section) of the sanitizing element (202).

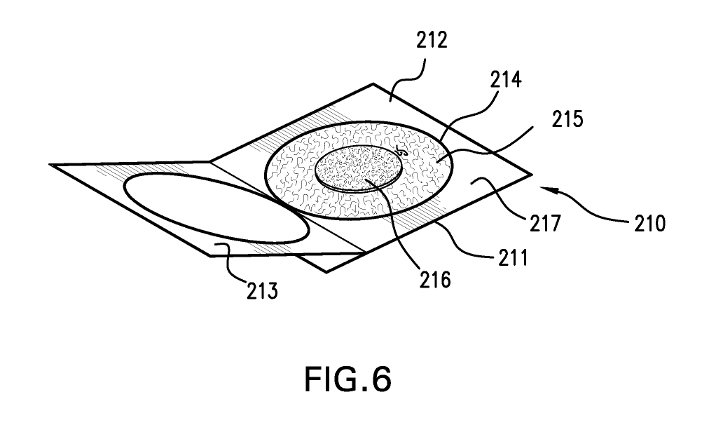

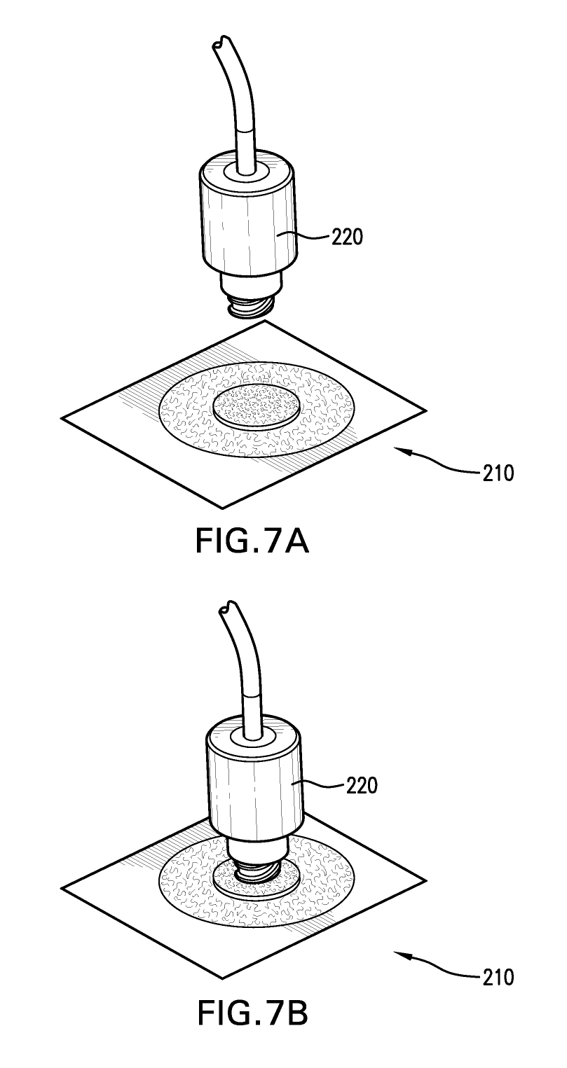

FIGS. 6 and 7 show different views of a sanitizing article according to the invention. The sanitizing article (210) has a container (211) comprised of first and second pieces (212, 213). A sanitizing element (214) is securely attached in the inner surface (217) of the first (lower) piece (212). In this embodiment the sanitizing element (214) is comprised of two layers (215, 216) (or a single layer of differing thickness when viewed in cross section). To use such a sanitizing article, the second (upper) piece (213) of the container (211) is peeled away from the first piece (212). FIG. 7 has two panels, A and B, which show a sanitizing article of the invention as depicted in FIG. 6 being brought into sanitizing association with a needless medical valve.

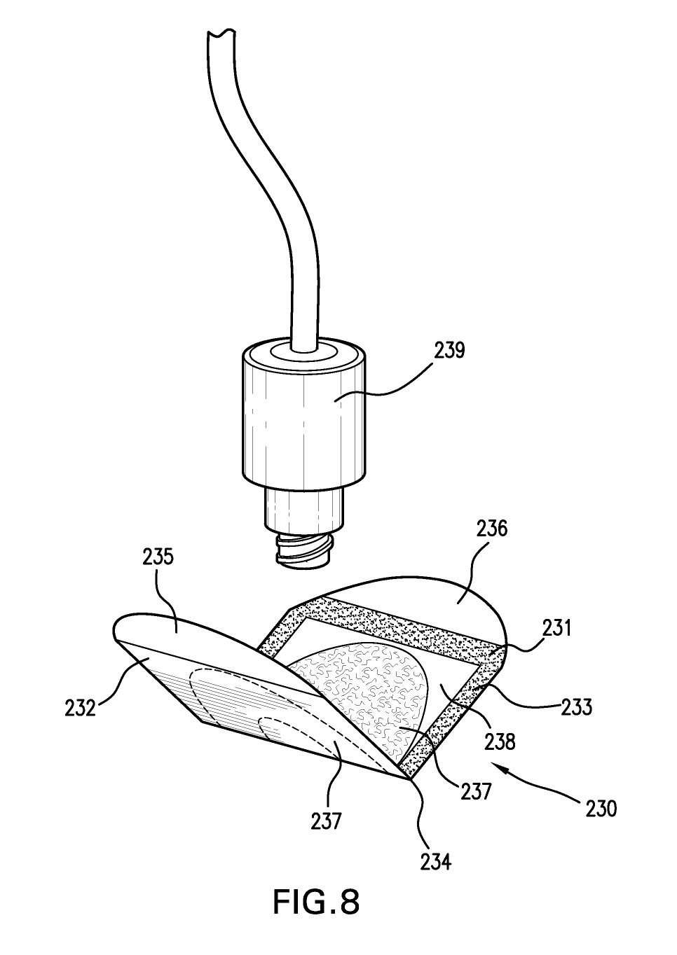

FIG. 8 shows a preferred partially folded, just-opened “butterfly” embodiment of a single-use sanitizing article according to the invention. In this figure the sanitizing article (230) has a container (231) comprised of first and second pieces (232, 233) folded from a single piece of material. Opposite the edges of the first and second pieces (232, 233) that comprise the fold (234), each of the first and second pieces has a distal edge region (235, 236) that is not attached to its counterpart as part of the sealing process in order to facilitate separation of the sealed portions of the container just prior to use. As those in the art will appreciate, a user can grasp the distal edge regions (235, 236) and by puling them apart cause the sealed portions of the container to come apart, exposing the sanitizing element (237), In the figure the remnants of the portions of the second piece (233) used to seal the second piece to the first piece (232) to form the sealed container are shown as hatched regions. A portion of the container that served as part of the internal cavity is shown as element 238, which does not have the sanitizing element (237) bound thereto. Also shown in the figure is a needless medical valve (239).

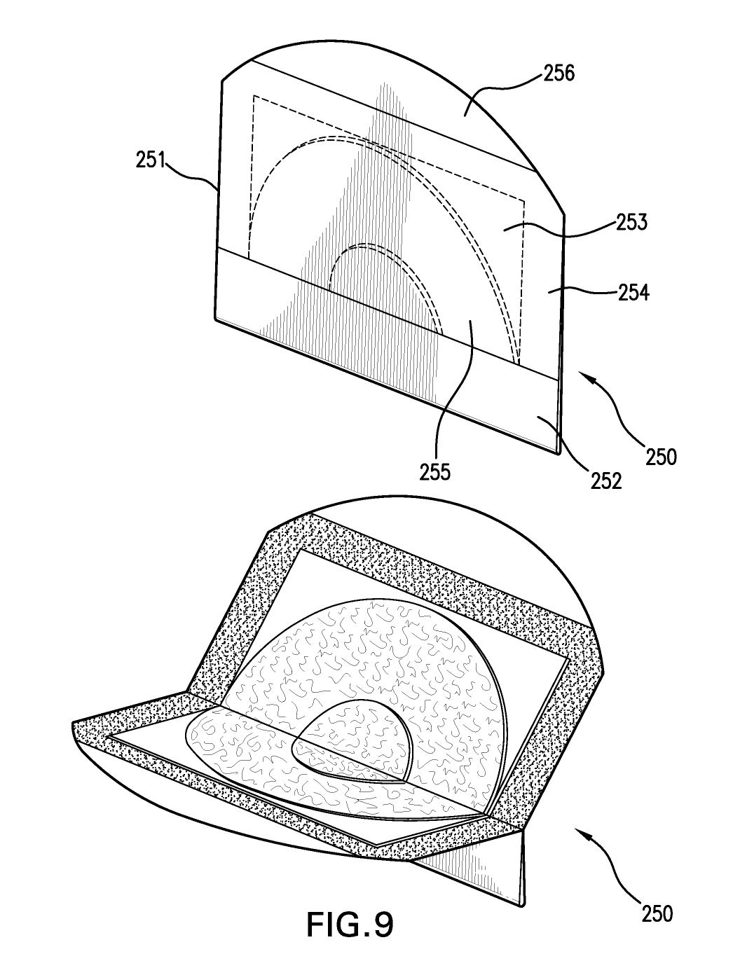

FIGS. 9 and 10 illustrate a particularly preferred embodiment of the invention. Each of these figures has two panels, A and B. FIG. 9 focuses on the sanitizing article (250), while FIG. 10 shows two views of the sanitizing article (250) in near sanitizing association with a needless medical valve (270). Turning the FIG. 9, panel A shows a sealed “butterfly”-type sanitizing article (250) wherein the container (251) is formed by folding a single piece of material so as to generate a device having a grasping element (252), a sealed internal cavity (253) (the seal along three sides of the cavity being indicated by hatching (254)) formed by joining first and second sections of the material to a sanitizing element (255) is then fixedly attached. The sanitizing element (255) in the depicted embodiment has a substrate formed from either (i) two concentrically positioned circular pieces of the same or different absorbent material each having a different diameter, with the smaller piece being attached to the upward facing surface of the larger bottom piece, or (ii) a single piece of absorbent material having a differing thickness when viewed in cross-section. Opposite the grasping element (252) is the “butterfly” portion (256) of the device to facilitate separation of the sealed portions of the container just prior to use. FIG. 9B shows the device of FIG. 9A after it has been opened, thereby exposing the sanitizing element (255) for use to sanitize a medical fitting such as a needleless valve. FIG. 10, panels A and B, shows the device of claim 9 being brought in sanitizing association with just such a needleless valve.

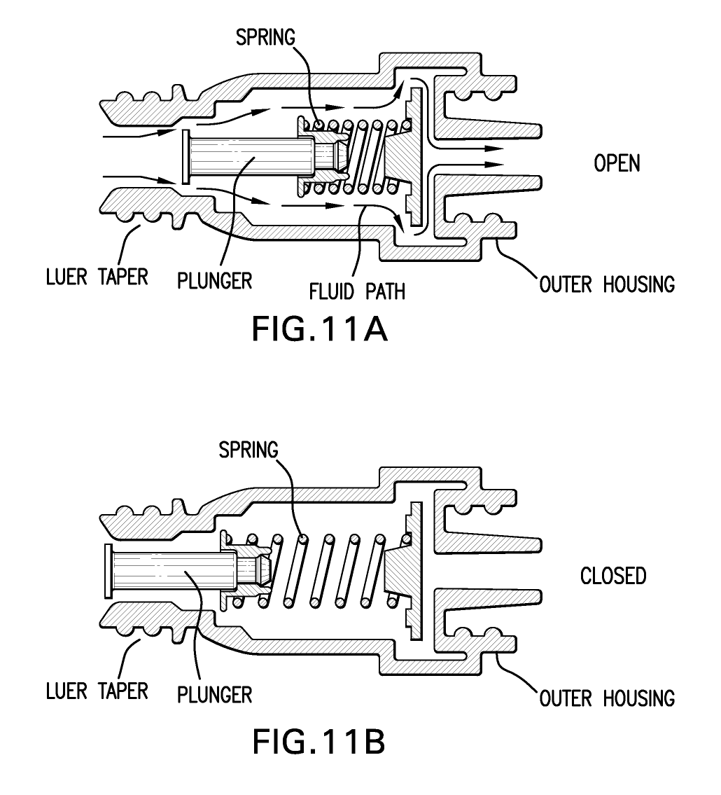

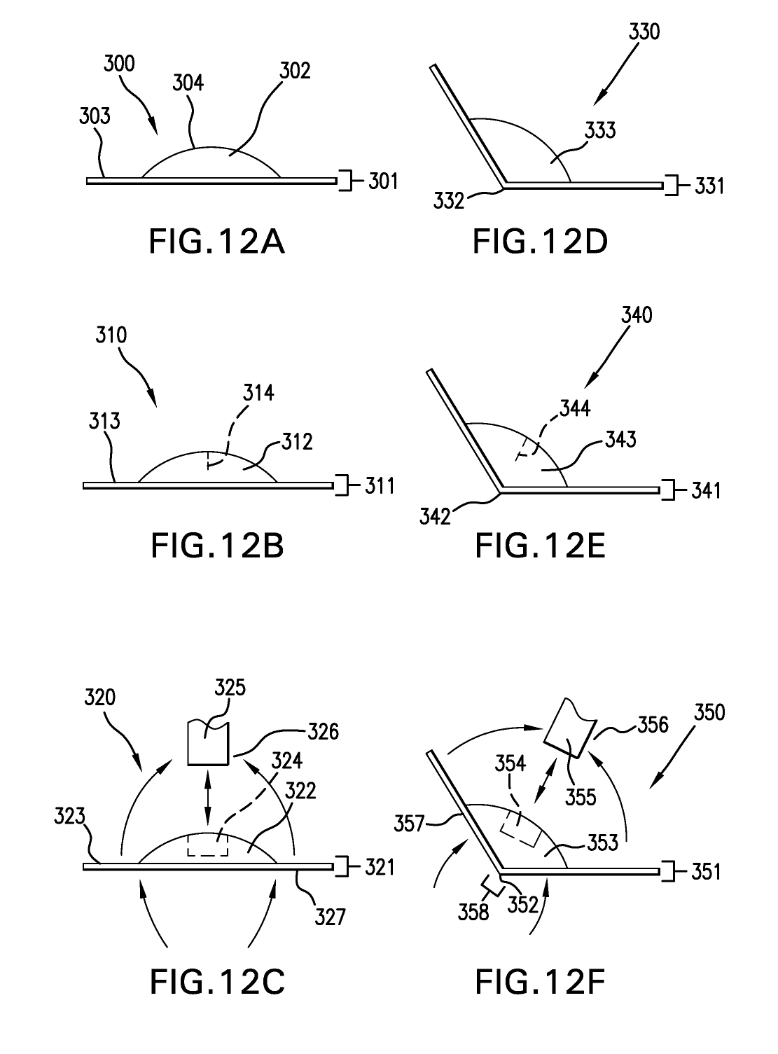

FIG. 12 shows six different representative embodiments (A-F; 300,310,320,330,340, 350) of sanitizing articles according to the invention. In this figure three of the embodiments (A, C, E; 300, 310, 320) represent sanitizing articles wherein the container comprises two pieces, an upper (first) piece (not shown) and a lower (second) piece (301, 311, 321) to which a sanitizing element (302, 312, 322) is secured to the inner surface (303, 313, 323) of the lower piece (301, 311, 321). In these embodiments (A, C, E; 300,310,320), the sanitizing element (302, 312, 322) is made to have a dome-like profile when the article is open, as shown. The sanitizing elements in these three embodiments (A, B, C) differ in that, in the embodiment illustrated in panel A, the sanitizing region is defined by a substantially smooth, curved upper surface (304) of the sanitizing element (302) that does not include a feature specifically designed to engage a structure on the medical fitting (e.g., a luer access device) to be sanitized. In contrast, in the embodiments depicted in panels B and C, the sanitizing region includes a feature (314, 324) designed to engage a structure (e.g., a luer taper; see FIG. 11) on a needleless medical valve. The feature in the embodiment of panel B is one or more slits or slots (314) machined or otherwise formed in the sanitizing element, whereas in the embodiment of panel C, the feature is a bore (324) that can engage the distal end (325) of, for example, a luer taper of a needleless medical valve (326) to facilitate sanitizing association between the sanitizing article and valve as the parts (320, 326) are brought together (represented by the double-headed arrow) and the sanitizing article (320) is collapsed (represented by curved arrows extending from the article (320) toward the valve (326)) around the valve by pressure by applied to the outer surface (327) of the of the sanitizing article (320) by a user holding the article (320) in one hand (not shown) and the valve (326) in the other hand (not shown). As will be appreciated, the substrates of such sanitizing elements (302, 312, 322) may be formed from a single a single piece of material (e.g., a surgical grade medical foam) or from different materials. When formed from different materials, the materials may be mixed; alternatively, layers of different materials can be used. As shown in FIG. 16, the sanitizing article (500) may also contain a protrusion (501) positioned in the central region of the lower layer (502) of the container (503; upper layer removed and not shown) and underlying the sanitizing element (504; here, comprised of two pieces, 505, 506) that serves to increase the pressure that can be applied to a corresponding medical fitting (e.g., the exposed surface of the plunger of a needleless medical valve (not shown)).

Panels D, E, and F of FIG. 12 show sanitizing article embodiments (330, 340, 350) wherein each container (331, 341, 351) is formed from one piece of material, typically by folding about an axis (332, 342, 352). In the embodiments shown in FIG. 12 panels D, E, and F, the sanitizing element (333, 343, 353) is made to have a dome-like profile when the article is open, as shown, and are similar to those (302, 312, 322) depicted in panels A, B, and C of this figure. As with the embodiments shown in panels B and C (310, 320), in the embodiments depicted in panels E and F, the sanitizing region includes a feature (344, 354) designed to engage a structure (e.g., a luer taper) on a needleless medical valve. The feature in the embodiment of panel E is one or more slits or slots (344) machined or otherwise formed in the sanitizing element, whereas in the embodiment of panel F, the feature is a bore (354) that can engage the distal end (355) of, for example, a luer taper of a needleless medical valve (356) to facilitate sanitizing association between the sanitizing article and valve as the parts (350, 356) are brought together (represented by the double-headed arrow) and the sanitizing article (350) is collapsed (represented by arrows below the article (350) and pointing upward toward the outer surface (357) of the article (350)) around the valve by pressure by applied to the outer surface (357) of the of the sanitizing article (350) by a user holding the article (350) in one hand (not shown) and the valve (356) in the other hand (not shown).

The embodiment shown in FIG. 12F also includes a grasping element (358), which can be formed in any suitable manner, including that as described above in conjunction with the embodiment shown in FIG. 4C.

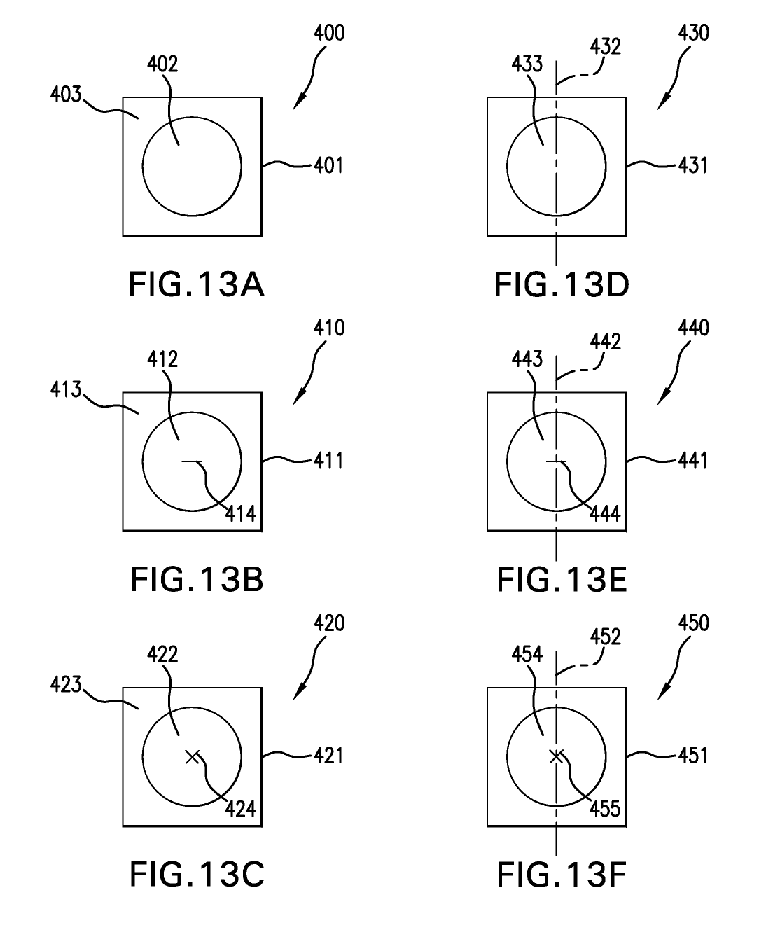

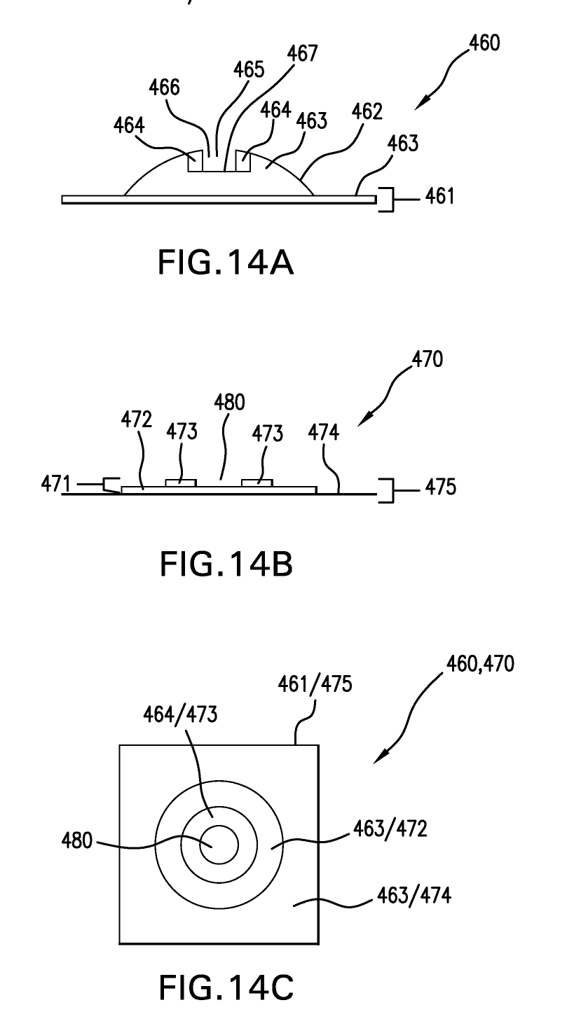

FIG. 13 shows six representative embodiments (A-F; 400, 410, 420, 430, 440, 450) of sanitizing articles according to the invention. In this figure three of the embodiments (panels A-C) represent sanitizing articles wherein the container comprises two pieces, an upper (first) piece (not shown) and a lower (second) piece (401, 411, 421) to which a sanitizing element (402, 412, 422) is secured to the inner surface (403, 413, 423) of the lower piece (401, 411, 421). In these embodiments (A-C), the sanitizing element (402, 412, 422) is made to have a dome-like profile, as is the case with the embodiments shown in FIG. 12. The sanitizing elements in the embodiments show in panels A-C of FIG. 14 differ with regard to features in the articles' respective sanitizing regions. In the embodiment illustrated in panel A, the sanitizing region is defined by a substantially smooth, curved upper surface that does not include a feature specifically designed to engage a structure on the medical fitting (e.g., a luer access device) to be sanitized, as is the case with the embodiment depicted in FIG. 12A. In contrast, in the embodiments depicted in panels B and C, the sanitizing region includes a feature (414, 424) designed to engage a structure (e.g., a luer taper; see FIG. 11) on a needleless medical valve. The feature in the embodiment of panel B is one slit or slot (414) machined or otherwise formed in the sanitizing element (412), whereas in the embodiment of panel C, the feature (424) is two intersecting slits or slots machined or otherwise formed in the sanitizing element (422) that can engage the distal end of, for example, a luer taper of a needleless medical valve (not shown) to facilitate sanitizing association between the sanitizing article and valve as the parts are brought together and the sanitizing article (420) is collapsed around the valve by pressure by applied to the outer surface of the of the sanitizing article by a user holding it in one hand and the valve in the other hand.

Panels D, E, and F of FIG. 13 show sanitizing article embodiments (430, 440, 450) wherein each container (431, 441, 451) is formed from one piece of material, typically by folding about an axis (432, 442, 452). In the embodiments shown in FIG. 13 panels D, E, and F, the sanitizing element (433, 443, 453) is formed in an analogous fashion to those depicted in panels A-C of this figure. The embodiment of panel D does not include a feature specifically designed to engage a structure on the medical fitting, whereas in panels E and F, such a feature (445, 455) is included.

FIG. 14 contains three panels, A-C, two of which (A and B) depict alternative sanitizing article embodiments. The embodiment (460) shown in cross-section in panel A comprises a container (461) to which a sanitizing element (462) is secured to its inner surface (463). Here, the sanitizing element (462) comprises two pieces (463, 464) which, when assembled, have a dome-like profile. A central bore (465) in the outer substrate component (463) serves to allow an inner substrate component (464) to be inserted and secured therein. In some embodiments, the inner substrate component itself can contain a cylindrical bore (466), although this is not a requirement. Also, the inner substrate component (464) may itself comprise a floor (not shown), such the bottom of the cylinder defined by the bore in the inner substrate component (464), when inserted into the outer substrate component, provides a surface for contacting an exposed surface of needleless medical valve (e.g., the outer, exposed surface of the top of the plunger of the valve portion of a conventional luer access device). In other embodiments, including the one depicted in panel A, a portion of the upper surface (467) of the bottom of the central bore (465) in the outer substrate component (463) is available for contact with an exposed surface if a medical fitting inserted into the sanitizing article. When the inner substrate component includes a bore, this can facilitate contact between the sanitizing region of the article and the various contoured external surfaces of the distal portion of a luer access device. In preferred embodiments, the inner and outer substrate components (463, 464) are made from different materials. Alternatively, the same materials can be used for the various inner and outer substrate components, although they may be oriented differently. For example, when the substrate components in such an embodiment are made from directionally oriented fibrous material, the fibers in one component (e.g., a central bore-containing cylindrical sleeve for insertion into a central core in an outer substrate component) may be oriented in a different direction, for example, offset by a desired angle (e.g., 30, 45, 60, or 90 degrees) to the fiber orientation in the other substrate component.

The embodiment (470) shown in cross-section in FIG. 14B is similar to that shown in FIG. 14A; however, the substrate (471) does not have dome-like profile and is instead comprised of two layers (472, 473), which may be comprised of the same or different materials disposed in the same or different orientations. In the depicted embodiment (470), the top substrate layer (473) is shaped like a washer (circular with a central bore; 480) and is comprised of an oriented fiber material. It is smaller than and concentrically positioned above and secured to the lower substrate layer (472), which is secured to the inner surface (474) of the container (475).

The illustration in FIG. 14C is a top view of the articles (460, 470) depicted in panels A and B.

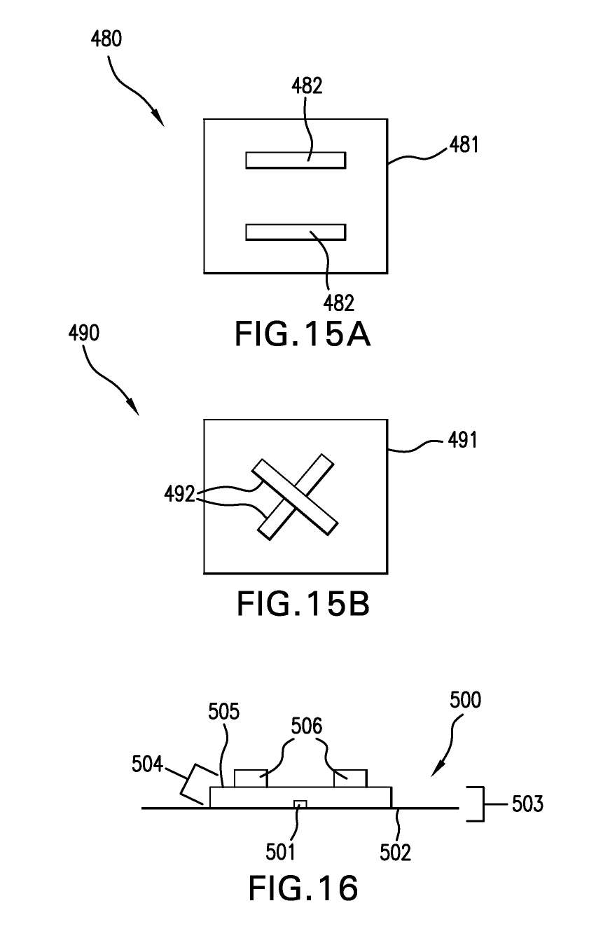

FIG. 15 concerns still other representative sanitizing article embodiments. Specifically, panels A and B depict two embodiments (480, 490) wherein the containers (481, 491) include preferably resilient structural members (482, 492) to stiffen or otherwise support the container of the particular article. One or more such structural members or supports can be included in a container in any desired configuration, preferably during container assembly or manufacture, and may include features to facilitate their bending or other deformation in one or more particular locations and/or directions.

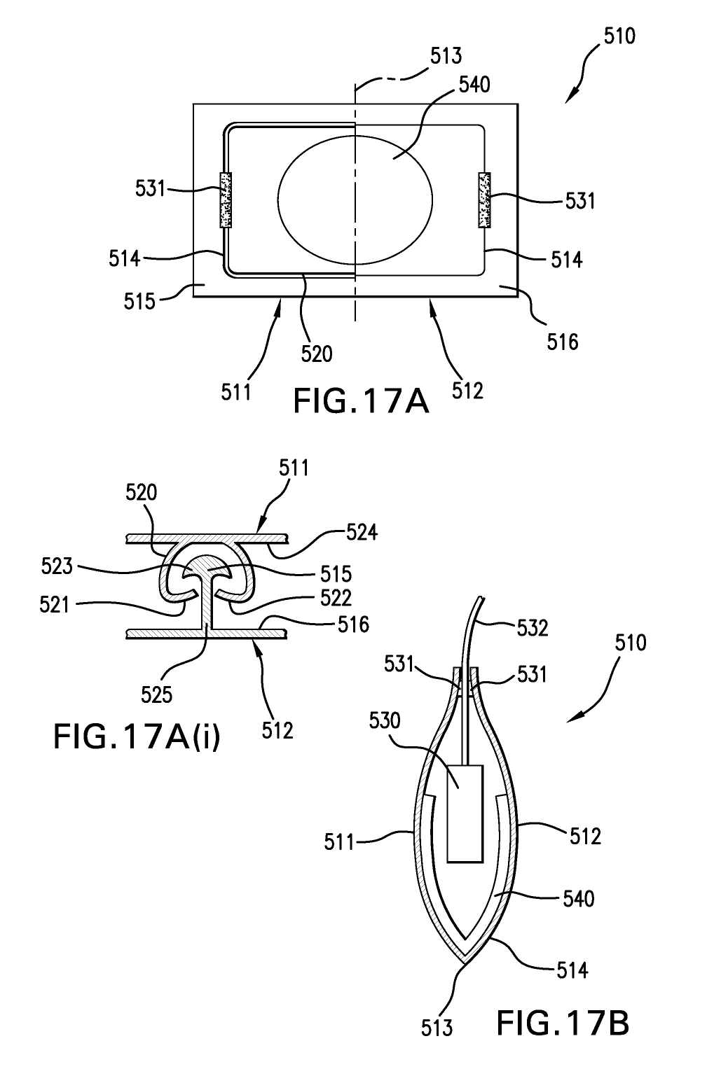

FIG. 17 has two panels, A and B, showing a sanitizing article embodiment (510) wherein different halves (511, 512, foldable about axis 513) of the container (514) can be temporarily sealed around a luer access (530) or other similar device after being used to sanitize the device. As shown in the figure, the inner surfaces of the container halves (511, 512) can include a sealing feature (514) that allows the halves (511, 512) to be sealed together. The sealing feature (514) is disposed (e.g., by bonding, co-extrusion, etc.) between the periphery of the sanitizing element (540) and the edges of the container (514). The sealing feature (514) can be any suitable structure, or combination of structures, that allows easy, preferably easily reversible, mating of the container halves. One representative example of such a sealing or closure feature is a Ziploc®-type system in which the inner surface (515) of one container half (511) has a C-shaped channel (520; see also inset 17-A(i)) disposed thereon such that the open side of the channel faces toward the inner surface (516) other container half (512) and allows the flanges (521, 522) of the channel (520) to engage hook-shaped features (522, 523 residing on a channel-complementary rail (525) positioned on the inner surface (516) of the other container half (512). After mating the container halves (511, 512), the sanitizing article can be left on the valve (530), where it can act as a cover to prevent inadvertent contact with the valve as well as to prevent or reduce subsequent recontamination of valve surfaces during periods when the valve is not being used to provide fluid access to the patient's vasculature. The access the valve (530), the sealing feature (514) components can be disengaged by gently pulling the container halves (511, 512) apart. In the embodiment shown, the sealing feature also includes a foam pad (531) in disposed in the midsections of the C-channel (520) and rail (525) to allow tight sealing of the sanitizing article about the fluid line (532) that extends from the proximal end of the valve (530).

### EXAMPLES

The invention will be better understood by reference to the following Examples, which are intended to merely illustrate certain aspects and embodiments of the invention. The scope of the invention is not to be considered limited thereto.

### Example 1

### Analysis of Contaminated Needleless Medical Valves Following Sanitizing Treatment

This example describes an assay for testing the effectiveness of using a sanitizing article according to the invention to sanitize a needleless medical valve contaminated with a bacterial biofilm.

The assay begins by inoculating a needleless medical valve with an aliquot of an inoculum containing a viable microorganism, preferably one encountered in typical hospital settings. For example, an aliquot from a log phase liquid culture of Geobacillus stearthermophilus can be inoculated directly onto the surface of the access port of each of several Smartsite® needleless medical valves (B. Braun Medical Inc., Bethlehem, Pa.). In addition, an aliquot from the same culture can also be inoculated directly onto the luer threads of some or all of the Smartsite® valves. The valves are then left undisturbed for a suitable period, for example, 30 minutes, at a temperature that promotes survival of the inoculated organisms (e.g., 35-37° C., although the temperature used may vary depending upon the particular microorganism being in the study). For each of the different device classes to be sanitized with an article according to the invention or a control device, e.g., a conventional IPA-saturated pad or another article against whom sanitizing performance is to be evaluated, four or five contaminated valves are preferably used. Several Smartsite® valves also are inoculated with the same amount of the G. stearthermophilus inoculum to serve as positive, untreated (in terms of sanitizing action) controls. One or more additional Smartsite® valves that have not been inoculated can be used as negative, uncontaminated controls.

After the desired period of incubation (e.g., 30 minutes), each of the test and control valves is sanitized as follows using a sanitizing article according to the invention or a conventional sterile cleansing pad saturated with a 70% isopropyl alcohol (IPA) (Webcol®, Kendall Co., Mansfield, Mass.). In each case, for those valves to under test that are to be exposed to sanitizing treatment, a sanitizing device is manually brought into contact with the previously inoculated surfaces of the access port and luer threads of a Smartsite® valve by gently pressing the device onto the valve. The sanitizing article is then rotated back and forth several times in relation to the valve, after which the sanitizing article is removed from contact with the valve and discarded. Each valve is then typically allowed to air dry in HEPA-filtered airflow for at least 30 seconds.

Following sanitizing treatment, under sterile conditions each of the valves under test is then transferred to a separate 100 mL beaker containing a small magnetic stir bar and 20 mL of sterile saline solution (1×PBS, 137 mM NaCl, 10 mM sodium phosphate, 2.7 mM KCl, pH 7.4). Each beaker is then placed on a stir plate and the valve-plus-solution is stirred slowly for a sufficient period (e.g., 2 minutes) to dislodge viable microorganisms from the valve into solution. Microorganisms are then collected from the solutions, for example, by filtering each solution through a separate 0.45-micron membrane filter. The filters can then be placed on fresh growth medium-containing plates, followed by incubation at an appropriate temperature for a sufficient period to allow growth of colonies sufficiently large to detect. After the incubation, colonies are counted to determine the number of colony forming units (CFUs) in each filtrate. The plates for the positive control valves (including any replicates) should have the largest numbers of colonies. The negative, uncontaminated control plates would be expected to have 0 colonies. Together, such results can be used to determine how well sanitizing articles according to the invention function. Such results can also be used to compare the sanitizing capabilities of the articles of the invention to other devices, be they conventional (e.g., 70% IPA wipes) or other, different designs. Reductions in contamination that exceed 2, 5, 10, 100, 10³3, 10⁴, 10⁵, 10⁶, or more -fold reductions (wherein each reduction by a factor of 10 is considered to be a “1 log reduction”, and so on) can be detected using these, or other suitable, methods. As will be appreciated, sanitizing articles that can markedly reduce, and preferentially completely eliminate, contamination by microorganisms introduced onto exposed surfaces near or in the path fluids must traverse to enter a needleless medical valve, are preferred.

### Example 2

### Assay for Assessing Effectiveness of Sanitizing Contaminated Needleless Valves

This example describes an assay for testing the effectiveness of sanitizing a needleless medical valve contaminated with a bacterial biofilm. This assay is similar to that described in Example 1, the difference being that after the contaminated needleless medical valves are disinfected, they are individually placed in a sterile chamber (e.g., a plastic 90 mm Petri dish) and allowed to incubate at a suitable temperature for a sufficient period. The incubation period is intended to allow contaminating microorganisms that remain on the contaminated but sanitized surface(s) to recover before being collected onto a 0.45 micron filter and transferred to a plate containing nutrient agar for outgrowth and CFU enumeration.

### Example 3

### Visual Assay for the Assessing Effectiveness of Sanitizing Contaminated Needless Valves

This example describes an assay for testing the effectiveness of sanitizing a needleless medical valve contaminated with a microorganism engineered to fluoresce under ultraviolet light. Procedures such as those described in this example can also be used to compare the sanitizing effect of articles according to the invention as compared to other valve-cleaning techniques.

This assay relies on applying approximately 100 uL of Glo Germ™ (Glo Germ™ Co., Moab, Utah) to the surface o the access port and luer threads of each of two or more ULTRASITE® needleless medical valves (B. Braun Medical Inc., Bethlehem, Pa.). Post-inoculation, each valve is photographed under ultraviolet light. Each valve under test is then sanitized using a test or control device using a suitable procedure, for example, a procedure such as described in Example 1 or 2, above. After sanitizing, each valve is again photographed under ultraviolet light. The results can then be compared to assess the sanitizing efficacy of particular article/valve/process combinations.

All of the compositions, articles, and methods described and claimed herein can be made and executed without undue experimentation in light of the present disclosure. While the, articles and methods of this invention have been described in terms of preferred embodiments, it will be apparent to those of skill in the art that variations may be applied to the articles, methods, and compositions without departing from the spirit and scope of the invention. All such variations and equivalents apparent to those skilled in the art, whether now existing or later developed, are deemed to be within the spirit and scope of the invention as defined by the appended claims.

All patents, patent applications, and publications mentioned in the specification are indicative of the levels of those of ordinary skill in the art to which the invention pertains. All patents, patent applications, and publications are herein incorporated by reference in their entirety for all purposes and to the same extent as if each individual publication was specifically and individually indicated to be incorporated by reference in its entirety for any and all purposes.

The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A patentable single-use sanitizing article configured to sanitize a needleless valve of a medical fitting, comprising a container comprising a first layer joined to a second layer to form a sealed internal cavity, wherein the container is adapted for manual opening to expose a sanitizing element attached to an inner surface of at least the first and/or second layer of the container, wherein the sanitizing element comprises a substrate and a sanitizing reagent dispersed in the substrate prior to use, wherein the substrate has a sanitizing region configured to engage in sanitizing association an accessible surface of a valve stem of a needleless valve of a medical fitting, which needleless valve optionally comprises a threaded valve body adapted to engage a complementary threaded portion of a fluid delivery device.

2. An article according to claim 1, wherein the substrate of the sanitizing element comprises an absorbent material selected from the group consisting of a naturally occurring material and a synthetic material, wherein when the substrate is a naturally occurring material, the naturally occurring material is optionally a natural sponge, and wherein when the substrate is a synthetic material, the synthetic material is optionally selected from the group consisting of a fibrous composition, a foam, and a gel.

3. An article according to claim 1, wherein the sanitizing reagent is a liquid formulation, optionally an aqueous solution.

4. An article according to claim 1, wherein the sanitizing reagent comprises a sanitizing compound selected from the group consisting of an alcohol, chlorhexidine, hydrogen peroxide, iodine, and silver ions.

5. An article according to claim 1, wherein at least one of the first layer and second layer is comprised of a laminated foil.

6. An article according to claim 1, wherein an outer surface of the first layer and/or second layer is labeled.

7. A kit comprising a plurality of articles according to claim 1.

8. A patentable method of sanitizing an accessible surface of a needleless medical valve, comprising contacting an accessible surface of the valve with a single-use sanitizing article according to claim 1 to sanitize the accessible surface, thereby sanitizing the accessible surface of the valve.

9. A method according to claim 8 wherein sanitizing of the accessible surface of the needleless medical valve results in at least a 10-fold reduction in microorganism contamination on the accessible surface, optionally more than a 100-fold, a 10³-fold, a 10⁴-fold, a 10⁵-fold, a 10⁶-fold, or 10⁷-fold reduction in microorganism contamination on the accessible surface.

10. A patentable method of reducing infection risk in a patient connected to a venous catheter having at least one medical fitting having a needleless valve, comprising contacting an accessible surface of a needleless medical valve of the venous catheter with a single-use sanitizing article according to claim 1 so as to sanitize the accessible surface of a valve, thereby reducing infection risk in the patient.
